(19) United States
(12) Patent Application Publication
(10) Pub. No.: US 2022/0298514 A1
(43) Pub. Date: Sep. 22, 2022

(54) AAV-ZYME AND USE FOR INFUSION REPLACEMENT THERAPY

(71) Applicant: AAV Gene Therapeutics, Inc., Beverly, MA (US)

(72) Inventor: Senthil Ramu, Boston, MA (US)

(21) Appl. No.: 17/641,754

(22) PCT Filed: Sep. 10, 2020
(86) PCT No.: PCT/US2020/050102

§ 371 Date: Mar. 9, 2022

Related U.S. Application Data
(60) Provisional application No. 62/898,801, filed on Sep. 11, 2019.

(51) Int. Cl. C12N 15/62; C12N 15/86; C07K 14/76; C07K 14/75; C07K 14/47

(52) U.S. Cl. CPC C12N 15/625; C12N 15/86; C07K 14/76; C07K 14/75; C07K 14/4717; C07K 2319/02; C12N 2750/14143; C12N 2830/008

(57) ABSTRACT

The invention described herein provides methods and compositions for using recombinant AAV-based viral vectors to express any gene-of-interest (GOI), preferably from muscle and/or liver tissues, for secretion of the polypeptides encoded by the GOI into the bloodstream, thus serving as an alternative treatment regimen for diseases caused by or characterized by lack of functional GOI, or for disease caused by or characterized by a pathological antigen that can be neutralized by a neutralizing peptide encoded by the GOI, such as an immunoglobulin, antibody, or antigen-binding fragment thereof. The method/composition of the invention can be used as an alternative for enzyme replacement therapy (ERT), or for antibody-based therapy.

### REFERENCE TO RELATED APPLICATIONS

The application claims the benefit of the filing date, under 35 U.S.C. 119(e), of U.S. provisional application No. 62/898,801, filed on Sep. 11, 2019, the entire content of which is incorporated herein by reference

### BACKGROUND OF THE INVENTION

The concept of enzyme replacement therapy (ERT) for lysosomal storage diseases was enunciated by de Duve in 1964 (Deduve, From Cytases to Lysosomes. Fed. Proc. Sep.-Oct.; 23:1045-1049, 1964). A model system, consisting of cultured skin fibroblasts from patients with mucopolysaccharidoses (MPS), showed that their defective glycosaminoglycan catabolism could be corrected by factors derived from cells of a different genotype. The corrective factors were identified as lysosomal enzymes with a special feature, or recognition signal, that would permit efficient uptake. A second model system was the clearance, in vivo, of lysosomal enzymes from plasma. The clearance was shown to be mediated by the mannose receptor of the reticuloendothelial system. This second system was immediately put to use for the treatment of Gaucher disease type I, in which macrophages are the affected cells. Later, recombinant lysosomal enzymes containing the mannose-6-phosphate (M6P) signal have been developed into pharmaceuticals for the treatment of Fabry disease, MPS I, MPS II, MPS VI and Pompe disease.

Despite the tremendous recent advancement in ERT, the enormous cost associated with such treatments remains a huge hurdle for patients suffering from diseases otherwise treatable by ERT. For example, Genzyme markets alglucosidase alfa as Myozyme. In 2006, the U.S. Food and Drug Administration (FDA) approved Myozyme as a suitable ERT treatment for children. Some health plans have refused to subsidize Myozyme for adult patients because it lacks approval for treatment in adults, as well as its high cost (US$300,000 per year for the life of the patient). On Aug. 1, 2014 the U.S. Food and Drug Administration announced the approval of Lumizyme (alglucosidase alfa) for treatment of patients with infantile-onset Pompe disease, including patients who are less than 8 years of age. Lumizyme was ranked in 2015 as the costliest drug per patient, with an average charge of $630,159 per year per patient.

In addition, most (if not all) ERT therapies are administered through inconvenient and potentially painful intraveneous (IV) infusions, at a medical facility such as hospital and clinics with proper equipments. Such treatments are usually required to be repeated, for the life span of the patients, some of which are very young patients.

Furthermore, ERT is susceptible to the development of immunity. Patients often develop neutralizing antibodies to the enzymes. As an example, recombinant (e.g., lab-made) human GAA (rhGAA) was approved as enzyme replacement therapy for Pompe disease patients in 2006. However, patients often develop immune tolerance to this therapy, in the form of anti-rhGAA antibodies that make the therapy less effective.

Thus there remains a need for additional or alternative therapies to treat diseases currently or otherwise treatable by ERT.

### SUMMARY OF THE INVENTION

In one aspect, the invention provides a recombinant adeno-associated viral (rAAV) vector, comprising: (1) a polynucleotide encoding a secretary signal peptide fused N-terminal to a protein encoded by a gene of interest (GOI); and, (2) a pair of inverted terminal repeat (ITR) flanking the polynucleotide; wherein the rAAV vector has a serotype or peusotype for preferential infection of liver or skeletal muscle tissue.

In certain embodiments, the secretary signal peptide comprises more than 50% or 60% of hydrophobic amino acids (e.g., about 20%, 30%, 40%, or 50% Leu).

In certain embodiments, the secretary signal peptide comprises 5, 6, 7, 8, 9, 10, 11, 12, 13, or 14 Leu residues.

In certain embodiments, the secretary signal peptide is the secretary signal peptide of an albumin, a globulin, or a fibrinogen.

In certain embodiments, the secretary signal peptide is the secretary signal peptide of ApoB (e.g., SEQ ID NO: 1).

In certain embodiments, the rAAV vector expresses in vivo more than 1 μg of plasma protein/dL of blood.

In certain embodiments, the serotype is AAV1, AAV6, AAV7, AAV8, or AAV9.

In certain embodiments, the serotype is AAV7, AAV8, or AAV9.

In certain embodiments, the pseudotype is AAV2/1, AAV/2/6, AAV2/7, AAV2/8, or AAV2/9.

In certain embodiments, the polynucleotide further comprises a 5′-UTR coding region, a 3′-UTR coding region, and a promoter sequence.

In certain embodiments, the promoter sequence is a liver- or hepatic cell-specific promoter and the serotype or peusotype is for preferential infection of liver.

In certain embodiments, the promoter sequence is a skeletal muscle-specific promoter and the serotype or peusotype is for preferential infection of skeletal muscle.

In certain embodiments, the skeletal muscle-specific promoter sequence comprises the AAVGTX sequence (SEQ ID NO: 20).

In certain embodiments, the GOI encodes protein deficient in a genetic disease or disorder in a host, and wherein expression of the GOI in the host alleviates the disease or disorder.

In certain embodiments, the genetic disease or disorder is Pompe disease or GAA deficiency, and the GOI encodes α-glucosidase or alglucosidase alfa.

In certain embodiments, the genetic disease or disorder is Fabry disease or a deficiency of α-galactosidase A (α-Gal A), and the GOI encodes α-galactosidase A.

In certain embodiments, the genetic disease or disorder is Gaucher disease or beta-glucocerebrosidase deficiency, and the GOI encodes beta-glucocerebrosidase.

In certain embodiments, the genetic disease or disorder is Hunter syndrome or MPS-II, and the GOI encodes lysosomal enzyme iduronate-2-sulfatase.

In certain embodiments, the genetic disease or disorder is hypophosphatasia (HPP), such as perinatal/infantile- and juvenile-onset HPP, and the GOI encodes asfotase alfa.

In certain embodiments, the genetic disease or disorder is lysosomal acid lipase deficiency (LAL-D), and the GOI encodes lysosomal acid lipase (LAL) or sebelipase alfa.

In certain embodiments, the genetic disease or disorder is mucopolysaccharidosis (MPS), such as MPS type I, and the GOI encodes alpha-L-iduronidase (IDUA).

In certain embodiments, the genetic disease or disorder is maroteux lamy syndrome (MPS VI), and the GOI encodes arylsylfatase B (ARSB).

In certain embodiments, the GOI encodes an immunoglobulin or an antigen-binding fragment thereof that neutralizes an antigen in a host, and wherein neutralization of the antigen alleviates a symptom or a cause of a disease or condition.

In certain embodiments, the antigen is TNFα, wherein the GOI encodes adalimumab, infliximab, golimumab, ustekinumab, exemptia, adfrar, amjevita, cyltezo, idacio, inflectra, remsima, infimab, inflectra (infliximab-dyyb), renflexis (infliximab-abda), flixabi, certolizumab pegol/CDP870, etanercept, and/or wherein the disease or disorder is rheumatoid arthritis, psoriatic arthritis, ankylosing spondylitis, Crohn's disease, ulcerative colitis, psoriasis, hidradenitis suppurativa, uveitis, and juvenile idiopathic arthritis.

In another aspect, the invention provides a pharmaceutical composition comprising the rAAV vector of the invention.

In yet another aspect, the invention provides a cell infected with the rAAV vector of the invention.

In still another aspect, the invention provides a recombinant AAV virus comprising the rAAV vector of the invention, wherein the serotype or pseudotype of the recombinant AAV virus is for preferential infection of liver or hepatic-tissue, or for preferential infection of skeletal muscle tissue.

In a further aspect, the invention provide a method of treating a genetic disease or disorder in a subject, the method comprising introducing the recombinant AAV virus of the invention into the subject.

In still further aspect, the invention provides a method of producing the rAAV vector of the invention, comprising introducing the rAAV vector of the invention into a packaging cell line that constitutively or inducibly provides rep/cap proteins in trans.

It should be understood that any one embodiment of any one of the aspects of the invention above, including specific embodiments described only in one section of the application, can be combined with one or more additional embodiments.

### BRIEF DESCRIPTION OF THE DRAWINGS

FIG. 1 is a schematic drawing (not to scale) showing a generic structure of the subject rAAV vector. The Gene of Interest (GOI) is fused in-frame and C-terminal to an exemplary (non-limiting) proprietary signal peptide sequence exhibiting enhanced secretion into the bloodstream/circulation—the proprietary secretion peptide ApoB. The coding sequence for the fusion peptide optionally is placed between a 5′-UTR and a 3′-UTR, under the control of a suitable promoter, preferably a tissue specific promoter, such as muscle-specific or liver-specific promoter. The entire recombinant sequence is flanked by two AAV ITR sequences, such as AAV9 or AAV8 ITR sequences for the preferred muscle and liver target expression, respectively.

FIG. 2 shows 6 exemplary rAAV vectors/constructs of the invention, in which the GOI is alpha-glucosidase, alpha-galactosidase, beta-glucocerebrosidase, iduronate-2-sulfatase, TNSALP, and lysosomal (acid) lipase.

FIG. 3 shows a schematic drawing (not to scale) for a representative plasmid construct containing an AAV vector (pAAV) with a gene of interest (GOI).

FIG. 4A shows DNA electrophoresis of the products after cleavages of the pAAV plasmids containing GOIs (drugs). FIG. 4B lists representative GOI genes and the sizes of the GOIs.

FIG. 5A shows healthy muscle cells after transduction with Fabrazyme- and Elaprase-expressing pAAV plasmids. FIG. 5B shows detection of Fabrazyme and Elaprase in the conditioned media of the transduced muscle cells by Western blotting.

### DETAILED DESCRIPTION OF THE INVENTION

The invention described herein provides methods and compositions for using recombinant AAV-based viral vectors to express any gene-of-interest (GOI), preferably from muscle and/or liver tissues, for secretion of the polypeptides encoded by the GOI into the bloodstream, thus serving as an alternative treatment regimen for diseases caused by or characterized by lack of functional GOI, or for disease caused by or characterized by a pathological antigen that can be neutralized by a neutralizing peptide encoded by the GOI, such as an immunoglobulin, antibody, or antigen-binding fragment thereof. The method/composition of the invention can be used as an alternative for enzyme replacement therapy (ERT), or for antibody-based therapy.

Thus in one aspect, the invention described herein provides a recombinant adeno-associated viral (rAAV) vector, comprising: (1) a polynucleotide encoding a secretary signal peptide fused N-terminal to a protein encoded by a gene of interest (GOI); and, (2) a pair of inverted terminal repeat (ITR) flanking the polynucleotide; wherein the rAAV vector has a serotype or peusotype for preferential infection of liver or skeletal muscle tissue.

Signal peptides are sometimes referred to as signal sequences, targeting signals, localization signals, localization sequences, leader sequences or leader peptides. These are short peptides present at the N-terminus of the majority of newly synthesized proteins that are destined towards the secretory pathway. Different signal peptides often show no sequence homology, but they all perform a similar, if not identical functions. Furthermore, many prokaryotic and eukaryotic signal peptides are interchangeable, suggesting conserved function during evolution. Mammalian signal peptides, including the secretary signal peptide of the invention, bind to the SRP (Signal Recognition Particle) which arrests elongation of the nascent polypeptide chain in the ribosome. Elongation arrest is released once the SRP is docked with the SRP receptor on the ER membrane, and elongation continues through a translocation machinery embedded in the ER membrane.

The secretary signal peptide of the invention is typically 16-30 amino acids in length. In certain embodiments, the N-terminus of the secretary signal peptide of the invention comprise a short positively charged stretch of amino acids of about, e.g., 1-5 residues. This short positively charged stretch of amino acids may comprise 2 to 3 Lys and/or Arg residues. In certain embodiments, the N-terminus of the secretary signal peptide of the invention does not comprise positively charged amino acids (e.g., Lys and/or Arg).

In certain embodiments, the core of the secretary signal peptide of the invention (e.g., immediately C-terminal to the positively charged residues, if present) contains a long stretch of hydrophobic amino acids (e.g., Leu, Ile, Val, Ala) of about, e.g., 5-16 residues, 8-13 residues, 9-12 residues, 10-14 residues, or 8-16 residues (such as 7, 8, 9, 10, 11, 12, 13, or 14 hydrophobic residues). In certain embodiments, this long stretch of hydrophobic amino acids form a single alpha-helix. In certain embodiments, the overall hydrophobicity of the secretary signal peptide of the invention is about 20%, 30%, 40%, 50%, 60%, or 70%. In certain embodiments, the secretary signal peptide of the invention has a core region having a stretch of poly-Leu, or about 6, 7, 8, 9, 10, 11, 12, 13, 14, 15, 16, 17, 18, 19 or 20 Leu residues. In certain embodiments, the poly-Leu stretch has about 8, 9, 10, 11, 12, 13, or 14 Leu residues, preferably 10 Leu residues. In certain embodiments, the secretary signal peptide of the invention has a core region having alternative Leu and hydrophobic non-Leu residues (such as Ile, Ala, Val), with a total length of about 6, 7, 8, 9, 10, 11, 12, 13, 14, 15, 16, 17, 18, 19 or 20 hydrophobic residues.

In certain embodiments, the secretary signal peptide of the invention has a stretch of polar amino acids (e.g., Thr, Ser, Gln, Asn) towards the C-terminus of the signal peptide. In certain embodiments, the stretch of polar amino acids comprises a sequence that is recognized and cleaved by a signal peptidase, or a cleavage site for a signal peptidase. In other embodiments, the secretary signal peptide of the invention does not have a signal peptidase cleavage site.

In certain embodiments, the secretary signal peptide of the invention comprises more than 50% or 60% of hydrophobic amino acids. In certain embodiments, the secretary signal peptide of the invention has about 20%, 30%, 40%, 50% Leu. In certain embodiments, about 20%, 30%, 40%, or 50% of the hydrophobic amino acids in the secretary signal peptide of the invention are Leu. In certain embodiments, the secretary signal peptide of the invention comprises 5, 6, 7, 8, 9, 10, 11, 12, 13, or 14 Leu residues.

In certain embodiments, the secretary signal peptide is the secretary signal peptide of an albumin, a globulin, or a fibrinogen.

In certain embodiments, the secretary signal peptide of the invention has the signal peptide sequence of ApoB, such as MDPPRPALLALLALPALLLLLLAGARA (SEQ ID NO: 1).

In certain embodiments, the secretary signal peptide of the invention has the signal peptide sequence of a serum albumin, such as MKWVTFISLLFLFSSAYS (SEQ ID NO: 2).

In certain embodiments, the secretary signal peptide of the invention has the signal peptide sequence of a fibrinogen, such as MFSMRIVCLVLSVVGTAWT (SEQ ID NO: 3).

In certain embodiments, the secretary signal peptide of the invention has the signal peptide sequence of a globulin, such as an Alpha 1 globulin, which may include al-antitrypsin, alpha 1-antichymotrypsin, orosomucoid (acid glycoprotein), serum amyloid A, and alpha 1-lipoprotein.

In certain embodiments, the secretary signal peptide of the invention has the signal peptide sequence of an Alpha 2 globulin, which may include haptoglobin, alpha-2u globulin, α2-macroglobulin, ceruloplasmin, thyroxine-binding globulin, alpha 2-antiplasmin, protein C, alpha 2-lipoprotein, angiotensinogen, and cortisol binding globulin.

All aforementioned globulin sequences are hereby incorporated herein by reference.

In certain embodiments, the secretary signal peptide of the invention has the signal peptide sequence of a (top 10) abundantly secreted protein in blood plasma, such as human blood plasma.

In certain embodiments, the secretary signal peptide of the invention has the signal peptide sequence of a (top 10) abundantly secreted protein from muscle, such as skeletal muscle.

In certain embodiments, the secretary signal peptide of the invention promotes secretion into the bloodstream when expressed in liver or skeletal muscle tissues.

In certain embodiments, the secretary signal peptide of the invention promotes secretion into the bloodstream in vivo at a level of more than 1 μg of plasma protein/dL of blood.

In certain embodiments, the serotype of the AAV is AAV1, AAV6, AAV7, AAV8, or AAV9. In certain embodiments, the serotype is AAV7, AAV8, or AAV9.

In certain embodiments, the pseudotype of the AAV is AAV2/1, AAV/2/6, AAV2/7, AAV2/8, or AAV2/9.

In certain embodiments, the polynucleotide further comprises a 5′-UTR coding region, a 3′-UTR coding region, and a promoter sequence.

In certain embodiments, the promoter sequence is a skeletal muscle-specific promoter and the serotype or peusotype is for preferential infection of skeletal muscle.

Muscle specific promoters are known in the art. See, for example, Wang et al., Construction and Analysis of Compact Muscle-specific Promoters for AAV Vectors, Gene Ther., 15(22): 1489-1499, 2008 (incorporated herein by reference). Examples of such promoters include the muscle creatine kinase (MCK) promoter or variants thereof, such as the dMCK (509-bp) promoter, the tMCK (720-bp) promoter, and the short, synthetic muscle promoter C5-12 (312-bp), all described in Wang (supra), with the first two being created by ligating a double or triple tandem of MCK enhancer (206-bp) respectively to its 87-bp basal promoter. Other variant promoters include the previously developed MCK-based enh358MCK (584-bp) and CK6 (589-bp) promoters. The dMCK and tMCK promoters are also essentially inactive in non-muscle cell lines as well as in the mouse liver (>200-fold weaker than the CMV promoter).

Another muscle-specific promoter that can be used in the rAAV vector of the invention is the human α-skeletal actin promoter (see, for example, McCarthy et al., Inducible Cre Transgenic Mouse Strain for Skeletal Muscle-specific Gene Targeting, Skelet. Muscle 2:8, 2012 (incorporated herein by reference).

Yet another muscle-specific promoter that can be used in the rAAV vector of the invention is the muscle-specific promoter within the Pitx3 gene that is situated between the first exon for eye and brain expression and exon 2 that contains the initiator ATG codon (see Coulon et al., A Muscle-specific Promoter Directs Pitx3 Gene Expression in Skeletal Muscle Cells, JBC 282:33192-33200, 2007, incorporated herein by reference).

Still another muscle-specific promoters that can be used in the rAAV vector of the invention include the muscle-specific promoter MHCK7, regulatory cassettes based on enhancer/promoter regions of murine muscle creatine kinase (CK) and α-myosin heavy-chain genes, as described in Salva et al., Design of Tissue-specific Regulatory Cassettes for High-level rAAV-mediated Expression in Skeletal and Cardiac Muscle, Mol. Ther. 15(2): 320-329, 2007 (incorporated herein by reference). MHCK7 (770 bp) directs high-level expression comparable to CMV and Rous sarcoma virus promoters in fast and slow skeletal and cardiac muscle, and low expression in the liver, lung, and spleen following systemic rAAV6 delivery in mice.

Further muscle-specific promoters that can be used in the rAAV vector of the invention include the muscle specific promoters derived from the genetic elements of the human slow isoform of troponin I gene (TnIS), as described in Zeng et al., Development of Strong Muscle-Specific Promoters for Gene Therapy of Duchenne Muscular Dystrophy, Musculoskeletal Gene and Cell Therapy: Muscular Dystrophies 16(supp. 1): S198, 2008 (incorporated herein by reference). The constructs contain one to four copies of the TnIS upstream enhancer (USE) or truncated USE (δUSE) fused to the minimal promoter of the TnIS gene, and had only very weak activity in non-muscle cells.

Further muscle-specific promoters that can be used in the rAAV vector of the invention include the synthetic muscle specific promoter SPc5-12, as described by Rasowo et al., Development of Novel Muscle-specific Adeno-Associated Viral Vector Constructs for Gene Therapy of Duchenne Muscular Dystrophy, Euro. Sci. J. 10(18): 23-37, 2014 (incorporated herein by reference).

Additional muscle-specific promoters that can be used in the rAAV vector of the invention are described in Sartorelli et al., Muscle-specific Gene Expression—A Comparison of Cardiac and Skeletal Muscle Transcription Strategies, Circulation Research 72(5): 925-931, 1993 (incorporated herein by reference), including cardiac or skeletal alpha-actin promoter, cardiac Troponin T (cTnT) promoter, Troponin C (TnC) promoter, myosin light chain-2 (MLC-2) promoter, cardiac myosin heavy chain (MHC) promoter (such as the beta-MHC promoter), muscle creatine kinase (MCK) promoter, and dystrophin promoter.

Further muscle-specific promoters that can be used in the rAAV vector of the invention include the proprietary muscle specific promoter AAVGTX. The sequence of the AAVGTX promoter is provided below.

(SEQ ID NO: 20)
ACCCTTCAGATTAAAAATAACTGAGGTAAGGGCCTGGGTAGGGGAGGTGG
TGTGAGACGCTCCTGTCTCTCCTCTATCTGCCCATCGGCCCTTTGGGGAG
GAGGAATGTGCCCAAGGACTAAAAAAAGGCCATGGAGCCAGAGGGGCGAG
GGCAACAGACCTTTCATGGGCAAACCTTGGGGCCCTGCTGTCCTCCTGTC
ACTAGCATGCCCCACTACGGGTCTAGGCTGCCCATGTAAGGAGGCAAGGC
CTGGGGACACCCGAGATGCCTGGTTATAATTAACCCAGACATGTGGCTGC
CCCCCCCCCCCCAACACCTGCTGCCTCTAAAAATAACCCTGTCCCTGGTG
GATCCCCTGCATGCGAAGATCTTCGAACAAGGCTGTGGGGGACTGAGGGC
AGGCTGTAACAGGCTTGGGGGCCAGGGCTTATACGTGCCTGGGACTCCCA
AAGTATTACTGTTCCATGTTCCCGGCGAAGGGCCAGCTGTCCCCCGCCAG
CTAGACTCAGCACTTAGTTTAGGAACCAGTGAGCAAGTCAGCCCTTGGGG
CAGCCCATACAAGGCCATGGGGCTGGGCAAGCTGCACGCCTGGGTCCGGG
GTGGGCACGGTGCCCGGGCAACGAGCTGAAAGCTCATCTGCTCTCAGGGG
CCCCTCCCTGGGGACAGCCCCTCCTGGCTAGTCACACCCTGTAGGCTCCT
CTATATAACCCAGGGGCACAGGGGCTGCCCTCATTCTACCACCACCTCCA
CAGCAC

In certain embodiments, the promoter sequence is a liver- or hepatic cell-specific promoter and the serotype or peusotype is for preferential infection of liver.

Liver specific promoters are also known in the art. See, for example, Kramer et al., In vitro and in vivo comparative study of chimeric liver-specific promoters, Mol. Ther. 7(3):375-385, 2003 (incorporated herein by reference), including α1-antitrypsin promoter with or without being linked to the albumin or hepatitis B enhancers, the Palb promoter, the Phpx promoter, and the PalAT promoter.

In certain embodiments, the GOI encodes protein deficient in a genetic disease or disorder in a host, and wherein expression of the GOI in the host alleviates the disease or disorder.

The recombinant AAV vector and recombinant AAV virus of the invention, together with the pharmaceutical composition comprising the same, can be used to treat a number of diseases and conditions, particularly diseases or indications treatable by enzyme replacement therapy.

Enzyme replacement therapy (ERT) refers to a medical treatment that replaces an enzyme that is deficient or absent in the body due to reasons such as genetic disorder. Typically, ERT is carried out by administering to the patient/host an intravenous (IV) infusion of a solution containing the enzyme. ERT does not correct the underlying genetic defect, but it increases the concentration of the enzyme that the patient is lacking. ERT is not limited to infusing enzymes, and can include infusing any functional version of a protein (including non-enzyme protein) that is lacking in the patient.

Diseases or conditions treatable by the AAV vector/virus/method of the invention are typically characterized by lacking a functional protein or enzyme, which diseases or indications can be alleviated (if not cured), treated, or prevented (e.g., at least the onset is delayed), by providing functional versions of the proteins or enzymes. The externally provided functional versions of the proteins or enzymes may be identical in sequence to the wild-type proteins or enzymes that are lacking in a host having such diseases or conditions, or may be a variant of the wild-type proteins or enzymes.

In certain embodiments, the diseases or conditions can be treated by infusing functional protein or enzyme to the host. Typically, the host is infused (e.g., injected intravenously) with the functional protein or enzyme periodically. For such diseases or conditions, periodic infusions can be avoided by introducing into the host the subject AAV virus that will infect target host tissues, such as liver and skeletal muscle, and stably express the encoded GOI throughout the life span of the host. The subject AAV virus may only need to be introduced into the host once, or can be introduced into the host multiple times as needed.

Representative polynucleotide sequences of the invention encoding the fusion of ApoB signal sequence and the gene-of-interest (GOI) protein are listed below. These polynucleotide sequences can be part of the subject AAV vector sequences. These sequences are for illustrative purpose only and are non-limiting.

In one embodiment, the GOI is α-glucosidase, and the disease or condition treatable by the subject AAV vector/virus is Pompe disease (also known as glycogen storage disease type II, or GAA deficiency), including late-onset (noninfantile) Pompe disease without evidence of cardiac hypertrophy in patients 8 years and older, as well as infantile-onset Pompe disease.

Pompe disease is an autosomal recessive metabolic disorder which is the first glycogen storage disease to be identified, in 1932, by the Dutch pathologist J. C. Pompe. Pompe disease is caused by an accumulation of glycogen in the lysosome due to deficiency of the lysosomal acid α-glucosidase enzyme, and it damages muscle and nerve cells throughout the body. It is the only glycogen storage disease with a defect in lysosomal metabolism. The build-up of glycogen causes progressive muscle weakness (myopathy) throughout the body, and affects various body tissues, particularly in the heart, skeletal muscles, liver and the nervous system.

An ERT for Pompe disease was first approved in the US as Myozyme (alglucosidase alfa, rhGAA). Myozyme is a biologically active recombinant human alglucosidase alfa produced in Chinese Hamster Ovary (CHO) cells, and is approved for administration by IV infusion of the solution. Myozyme treatment clearly prolongs ventilator-free survival and overall survival, and early diagnosis and early treatment lead to significantly better outcomes.

Another ERT for Pompe disease was approved by the FDA in 2010 as Lumizyme, a similar version of Myozyme with the same generic ingredient but different manufacturing process, for the treatment of late-onset Pompe disease.

The composition and methods of the invention can be used to produce alpha-glucosidase or alglucosidase alfa expressed permanently from AAV vectors stably maintained with the host's genome as extra chromosomal material, and secreted into a patient's blood stream, thus effectively treating Pompe disease.

A representative alpha-glucosidase fusion protein that can be encoded by the AAV vector of the invention is described below.

(SEQ ID NO: 4) [symbol]
AHPGRPRAVPTQCDVPPNSRFDC
APDKAITQEQCEARGCCYIPAKQGLQGAQMGQPWCFFPPSYPSYKLENLS
SSEMGYTATLTRTTPTFFPKDILTLRLDVMMETENRLHFTIKDPANRRYE
VPLETPHVHSRAPSPLYSVEFSEEPFGVIVRRQLDGRVLLNTTVAPLFFA
DQFLQLSTSLPSQYITGLAEHLSPLMLSTSWTRITLWNRDLAPTPGANLY
GSHPFYLALEDGGSAHGVFLLNSNAMDVVLQPSPALSWRSTGGILDVYIF
LGPEPKSVVQQYLDVVGYPFMPPYWGLGFHLCRWGYSSTAITRQVVENMT
RAHFPLDVQWNDLDYMDSRRDFTFNKDGFRDFPAMVQELHQGGRRYMMIV
DPAISSSGPAGSYRPYDEGLRRGVFITNETGQPLIGKVWPGSTAFPDFTN
PTALAWWEDMVAEFHDQVPFDGMWIDMNEPSNFIRGSEDGCPNNELENPP
YVPGVVGGTLQAATICASSHQFLSTHYNLHNLYGLTEAIASHRALVKARG
TRPFVISRSTFAGHGRYAGHWTGDVWSSWEQLASSVPEILQFNLLGVPLV
GADVCGFLGNTSEELCVRWTQLGAFYPFMRNHNSLLSLPQEPYSFSEPAQ
QAMRKALTLRYALLPHLYTLFHQAHVAGETVARPLFLEFPKDSSTWTVDH
QLLWGEALLITPVLQAGKAEVTGYFPLGTWYDLQTVPVEALGSLPPPPAA
PREPAIHSEGQWVTLPAPLDTINVHLRAGYIIPLQGPGLTTTESRQQPMA
LAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLL
MGEQFLVSWC

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of alpha-glucosidase (EC 3.2.1.20) has long been well-characterized as hydrolyzing terminal non-reducing (1→4)-linked alpha-glucose residues to release a single alpha-glucose molecule (see, e.g., Chiba, Molecular mechanism in alpha-glucosidase and glucoamylase. Biosci. Biotechnol. Biochem. 61(8):1233-1239, August 1997). Its activity can be measured using any commercially available kits, such as the Alpha-Glucosidase Activity Assay Kit (Colorimetric) (Cat. No. ab174093), marketed by Abcam (Cambridge, UK). Thus it is well within the skill of one of ordinary skill in the art to make conservative amino acid changes without significantly/adversely affecting the catalytic activity of alpha-glucosidase.

A polynucleotide sequence encoding the above alpha-glucosidase fusion is provided.

(SEQ ID NO: 5) [symbol]
ATGGACCCACCGCG
GCCAGCGTTGCTGGCTTTGCTCGCGCTTCCGGCACTTCTCTTGTTGCTGC
TTGCAGGTGCCCGCGCAGCGCATCCAGGGAGGCCACGGGCTGTACCGACG
CAGTGCGACGTGCCGCCAAACAGCCGGTTTGATTGTGCTCCCGACAAAGC
CATCACGCAAGAACAATGTGAAGCAAGGGGGTGCTGTTACATTCCCGCAA
AACAGGGACTGCAAGGTGCCCAAATGGGCCAACCCTGGTGCTTCTTTCCG
CCTAGCTACCCCTCCTACAAGCTCGAAAATCTTTCATCTAGCGAGATGGG
CTATACCGCGACTCTGACACGCACGACTCCTACGTTTTTCCCCAAGGACA
TACTTACACTGCGGTTGGATGTCATGATGGAGACCGAAAATAGATTGCAT
TTCACTATTAAAGACCCCGCAAACAGGCGGTATGAGGTTCCCCTCGAAAC
ACCTCACGTACACTCACGAGCACCAAGTCCGCTGTATAGCGTGGAGTTCA
GCGAGGAGCCGTTCGGGGTAATAGTAAGGAGACAACTTGACGGACGAGTT
TTGCTGAATACGACAGTCGCGCCGCTCTTCTTCGCAGATCAGTTCTTGCA
GTTGTCTACCTCACTTCCATCCCAATATATCACGGGCTTGGCGGAGCACC
TTTCTCCCCTCATGTTGAGCACTAGCTGGACACGGATCACCCTGTGGAAT
CGAGACCTTGCGCCGACTCCTGGGGCGAATCTGTACGGGTCTCATCCCTT
CTATTTGGCCCTCGAAGATGGGGGATCAGCGCACGGTGTTTTTCTGCTTA
ACAGTAACGCAATGGACGTAGTATTGCAACCGAGTCCAGCTTTGTCATGG
CGAAGCACAGGGGGGATCTTGGACGTCTACATATTTTTGGGCCCTGAGCC
CAAATCAGTCGTCCAACAATATCTCGATGTGGTAGGGTATCCCTTCATGC
CACCGTACTGGGGACTCGGGTTTCACCTTTGCCGATGGGGTTACTCAAGT
ACTGCGATAACACGACAGGTTGTTGAGAACATGACTCGCGCACATTTTCC
CCTGGACGTACAGTGGAACGACTTGGACTATATGGATTCCCGGCGCGACT
TTACCTTCAACAAGGATGGGTTTAGGGACTTCCCTGCGATGGTCCAGGAG
TTGCATCAAGGGGGGCGACGGTATATGATGATCGTAGATCCGGCAATTTC
TAGTTCTGGGCCTGCGGGGTCATACAGACCATATGATGAAGGGTTGCGGC
GGGGGGTATTTATTACAAACGAAACAGGGCAGCCACTTATTGGCAAAGTA
TGGCCGGGTTCTACGGCGTTCCCCGATTTCACCAATCCCACGGCGCTCGC
TTGGTGGGAAGATATGGTTGCCGAATTTCACGACCAGGTGCCTTTTGATG
GTATGTGGATAGACATGAATGAGCCAAGTAATTTCATCAGGGGGAGCGAG
GATGGGTGTCCCAACAATGAGTTGGAAAACCCACCGTACGTCCCGGGGGT
AGTCGGGGGTACTCTCCAGGCAGCCACGATATGTGCGTCATCACATCAGT
TTTTGAGTACGCATTATAATTTGCACAACTTGTACGGGTTGACTGAAGCA
ATCGCGAGCCACCGCGCCCTCGTGAAAGCCCGCGGGACTCGACCTTTTGT
CATCTCAAGATCAACCTTTGCGGGACACGGTCGCTACGCAGGACACTGGA
CCGGGGATGTCTGGTCCTCTTGGGAACAACTCGCGAGCTCAGTCCCTGAG
ATCCTCCAATTTAACCTGCTTGGCGTACCTCTCGTTGGTGCTGATGTCTG
TGGATTCTTGGGGAACACCAGCGAGGAGCTGTGTGTAAGGTGGACACAGC
TTGGAGCCTTCTATCCCTTCATGAGAAACCATAACAGCTTGTTGTCTTTG
CCCCAGGAGCCGTATAGCTTCAGTGAGCCCGCCCAGCAGGCAATGAGAAA
GGCGCTTACTCTGAGATACGCCTTGCTCCCACACCTGTACACACTCTTCC
ACCAGGCTCACGTTGCCGGAGAAACAGTCGCCAGGCCACTTTTTCTGGAG
TTTCCAAAAGATTCCAGTACGTGGACCGTCGACCACCAACTTTTGTGGGG
CGAAGCTCTCTTGATTACTCCAGTATTGCAAGCCGGTAAGGCTGAAGTGA
CGGGGTATTTTCCTCTGGGAACTTGGTATGATCTTCAAACTGTCCCTGTT
GAGGCCTTGGGTTCCCTCCCGCCGCCGCCAGCCGCACCTAGAGAACCAGC
GATACACAGCGAAGGGCAATGGGTTACACTTCCGGCGCCCCTGGACACGA
TCAATGTGCACCTCCGGGCTGGGTATATTATTCCCCTTCAAGGGCCGGGA
CTTACTACCACCGAATCACGGCAACAGCCGATGGCCCTGGCGGTTGCACT
TACCAAGGGTGGGGAGGCTAGAGGGGAACTGTTCTGGGATGACGGAGAAT
CTCTGGAGGTGTTGGAGCGAGGGGCTTACACTCAAGTCATATTTCTTGCT
AGAAATAACACCATAGTCAACGAGTTGGTGCGGGTGACGTCCGAGGGTGC
CGGATTGCAGTTGCAGAAAGTCACCGTGCTGGGTGTGGCCACGGCTCCAC
AGCAAGTTCTCTCAAATGGTGTGCCCGTAAGCAACTTTACTTACAGCCCG
GACACAAAGGTTCTTGATATATGTGTGTCACTCTTGATGGGGGAGCAGTT
TTTGGTTAGTTGGTGCCATCATCACCACCACCATTAG

[symbol]

[symbol]

The double underlined sequence at the 5′-end and the bold italic sequence at the 3′-end represent end tag sequences that can be used for manufacturing/amplification of the coding sequences in the middle.

In one embodiment, the GOI is α-galactosidase, and the disease or condition treatable by the subject AAV vector/virus is Fabry disease (also known as Anderson-Fabry disease).

Fabry disease is a rare X-linked genetic disease that also belong to the group of lysosomal storage diseases. The first descriptions of this condition were made simultaneously by the dermatologist Johannes Fabry and the surgeon William Anderson in 1898. It is caused by a genetic mutation in alpha-galactosidase A (a-GAL A, encoded by the GLA gene) that processes sphingolipids, leading to a glycolipid known as globotriaosylceramide (abbreviated as Gb3, GL-3, or ceramide trihexoside) to accumulate within the walls of blood vessels, as well as in other tissues and organs, and impairment of their respective functions. Fabry disease can affect many parts of the body, including the kidneys, heart, and skin.

The treatment for Fabry disease varies depending on the organs affected by the condition, and the underlying cause can be addressed by enzyme replacement therapy.

One ERT for Fabry disease is agalsidase alpha under the brand name Replagal which was granted marketing approval in the EU in 2001. Another ERT for Fabry disease is Fabrazyme (agalsidase beta, or Alpha-galactosidase) that was approved in 2003 by the FDA. Fabrazyme's annual cost was about US$200,000 per patient. Clinically, the two products are generally perceived to be similar in effectiveness.

The composition and methods of the invention can be used to produce α-galactosidase or agalsidase beta expressed permanently from AAV vectors stably maintained with the host's genome as extra chromosomal material, and secreted into a patient's blood stream, thus effectively treating Fabry disease.

A representative alpha-galactosidase fusion protein that can be encoded by the AAV vector of the invention is described below.

(SEQ ID NO: 6) [symbol]
LDNGLARTPTMGWLHWERFMCNL
DCQEEPDSCISEKLFMEMAELMVSEGWKDAGYEYLCIDDCWMAPQRDSEG
RLQADPQRFPHGIRQLANYVHSKGLKLGIYADVGNKTCAGFPGSFGYYDI
DAQTFADWGVDLLKEDGCYCDSLENLADGYKHMSLALNRTGRSIVYSCEW
PLYMWPFQKPNYTEIRQYCNHWRNFADIDDSWKSIKSILDWTSFNQERIV
DVAGPGGWNDPDMLVIGNFGLSWNQQVTQMALWAIMAAPLFMSNDLRHIS
PQAKALLQDKDVIAINQDPLGKQGYQLRQGDNFEVWERPLSGLAWAVAMI
NRQEIGGPRSYTIAVASLGKGVACNPACFITQLLPVKRKLGFYEWTSRLR
SHINPTGTVLLQLENTMQMSLKDLL

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of alpha-galactosidase (EC 3.2.1.22) has long been well-characterized as hydrolyzing the terminal alpha-galactosyl moieties from glycolipids and glycoproteins (see, e.g., Scriver et al., The Metabolic & Molecular Basis of Inherited Disease (8th ed.). McGraw-Hill. ISBN 978-0-07-913035-8, December 2000). Its activity can be measured using any commercially available kits, such as the Alpha-Galactosidase Activity Assay Kit (Cat. No. ab239716), marketed by Abcam (Cambridge, UK). Thus it is well within the skill of one of ordinary skill in the art to make conservative amino acid changes without significantly/adversely affecting the catalytic activity of alpha-galactosidase.

A polynucleotide sequence encoding the above alpha-galactosidase fusion is provided.

(SEQ ID NO: 7) [symbol]
ATGGACCCTCCACG
ACCAGCACTTCTGGCCTTGCTGGCACTCCCTGCCTTGCTTCTTTTGCTGC
TGGCCGGCGCTCGGGCACTCGACAATGGTCTGGCTCGCACTCCTACAATG
GGTTGGCTTCATTGGGAGCGATTCATGTGCAATCTGGACTGCCAGGAGGA
GCCAGACAGCTGCATTAGCGAGAAACTGTTCATGGAAATGGCAGAGCTTA
TGGTATCTGAAGGATGGAAAGACGCCGGGTACGAATATTTGTGTATAGAC
GATTGCTGGATGGCCCCACAGCGCGATTCTGAGGGCCGGCTTCAAGCTGA
CCCTCAGCGCTTCCCACACGGGATAAGGCAATTGGCTAATTACGTTCATT
CTAAGGGGCTTAAACTTGGAATCTACGCTGACGTGGGTAATAAAACGTGC
GCAGGATTCCCCGGTAGTTTCGGGTACTATGATATAGACGCGCAGACGTT
CGCCGACTGGGGGGTTGACCTTTTGAAGTTCGATGGATGCTATTGTGATA
GTCTCGAGAACCTTGCTGACGGATACAAACATATGAGCTTGGCTCTGAAC
CGGACAGGGCGATCAATCGTGTACTCTTGTGAATGGCCTCTGTACATGTG
GCCTTTTCAAAAGCCAAATTATACTGAAATACGGCAGTATTGTAATCATT
GGCGGAACTTCGCGGACATCGACGACTCCTGGAAATCCATAAAATCAATT
CTTGACTGGACCAGCTTCAATCAAGAGAGAATCGTTGATGTAGCCGGTCC
CGGAGGGTGGAATGACCCCGATATGCTGGTCATAGGCAACTTTGGATTGT
CATGGAACCAGCAAGTAACACAGATGGCTCTGTGGGCTATCATGGCTGCG
CCCTTGTTTATGTCAAACGACCTGAGACATATAAGTCCTCAAGCTAAGGC
GCTCCTCCAAGATAAGGACGTGATTGCTATAAACCAAGATCCCCTTGGAA
AGCAGGGATACCAACTTAGACAGGGTGATAACTTTGAGGTGTGGGAACGA
CCACTTAGCGGGCTGGCGTGGGCAGTCGCTATGATAAATCGGCAAGAAAT
TGGCGGGCCCCGCAGCTACACCATTGCGGTCGCCTCTCTGGGAAAGGGAG
TTGCGTGCAATCCGGCCTGCTTCATTACACAATTGCTCCCCGTCAAGCGC
AAACTTGGTTTTTACGAGTGGACGAGCAGATTGAGATCCCACATCAACCC
AACGGGGACAGTCCTTCTGCAGCTTGAGAATACGATGCAGATGAGCTTGA
AAGACCTCTTGCATCACCATCATCATCATTAG

[symbol]

The double underlined sequence at the 5′-end and the bold italic sequence at the 3′-end represent end tag sequences that can be used for manufacturing/amplification of the coding sequences in the middle.

In one embodiment, the GOI is β-glucocerebrosidase, and the disease or condition treatable by the subject AAV vector/virus is Gaucher's disease.

Gaucher's disease is named after the French physician Philippe Gaucher, who originally described it in 1882, and is the most common of the lysosomal storage diseases. It is a form of sphingolipidosis (a subgroup of lysosomal storage diseases), as it involves dysfunctional metabolism of sphingolipids.

Gaucher's disease is a genetic disorder caused by numerous recessive/autosomal mutation in the GBA gene located on chromosome 1, which encodes glucocerebrosidase (also known as beta-glucosidase or glucosylceramidase), resulting in accumulation of glucocerebroside (a sphingolipid, also known as glucosylceramide) in cells, particularly in white blood cells and especially in macrophages (mononuclear leukocytes). Glucocerebroside can also collect in the spleen, liver, kidneys, lungs, brain, and bone marrow. The disorder is characterized by bruising, fatigue, anemia, low blood platelet count and enlargement of the liver and spleen.

In all, about 80 known GBA gene mutations are grouped into three main types. For those with type-I and most type-III, enzyme replacement treatment with intravenous recombinant glucocerebrosidase can decrease liver and spleen size, reduce skeletal abnormalities, and reverse other manifestations. This treatment costs more than US$200,000 annually for a single person and should be continued for life.

An ERT for Gaucher's disease was first approved by the FDA in 1991 as alglucerase (Ceredase), which was a version of glucocerebrosidase that was harvested from human placental tissue and then modified with enzymes. Ceredase has since been withdrawn from the market due to the approval of similar drugs made with recombinant DNA technology instead of being harvested from tissue. Current available recombinant glucocerebrosidases include: Imiglucerase (Cerezyme, approved in 1995), Velaglucerase (VPRIV, approved in 2010), and Taliglucerase alfa (Elelyso, approved in 2012). Cerezyme is a recombinantly-produced analogue of the human β-glucocerebrosidase, and has an annual cost of $200,000 in the U.S. Velaglucerase alfa is a recombinant form of glucocerebrosidase indicated as a long-term enzyme replacement therapy for those suffering from Gaucher disease Type 1. It has an identical amino acid sequence to the naturally occurring enzyme. Taliglucerase alfa is a recombinant glucocerebrosidase and was the first plant-made pharmaceutical approved by the FDA. Elelyso was the third most costly pharmaceuticals in 2016, with an average cost of $483,242 per year per patient.

The composition and methods of the invention can be used to produce beta-glucocerebrosidase or expressed permanently from AAV vectors stably maintained with the host's genome as extra chromosomal material, and secreted into a patient's blood stream, thus effectively treating Gaucher's disease.

A representative beta-glucocerebrosidase fusion protein that can be encoded by the AAV vector of the invention is described below.

(SEQ ID NO: 8) [symbol]
ARPCIPKSFGYSSVVCVCNATYC
DSFDPPTFPALGTFSRYESTRSGRRMELSMGPIQANHTGTGLLLTLQPEQ
KFQKVKGFGGAMTDAAALNILALSPPAQNLLLKSYFSEEGIGYNIIRVPM
ASCDFSIRTYTYADTPDDFQLHNFSLPEEDTKLKIPLIHRALQLAQRPVS
LLASPWTSPTWLKTNGAVNGKGSLKGQPGDIYHQTWARYFVKFLDAYAEH
KLQFWAVTAENEPSAGLLSGYPFQCLGFTPEHQRDFIARDLGPTLANSTH
HNVRLLMLDDQRLLLPHWAKVVLTDPEAAKYVHGIAVHWYLDFLAPAKAT
LGETHRLFPNTMLFASEACVGSKFWEQSVRLGSWDRGMQYSHSIITNLLY
HVVGWTDWNLALNPEGGPNWVRNFVDSPIIVDITKDTFYKQPMFYHLGHF
SKFIPEGSQRVGLVASQKNDLDAVALMHPDGSAVVVVLNRSSKDVPLTIK
DPAVGFLETISPGYSIHTYLWRRQ

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of beta-glucocerebrosidase (EC 3.2.1.45) has long been well-characterized as hydrolyzing the beta-glucosidic linkage of the chemical glucocerebroside (see, e.g., Lieberman, A Guided Tour of the Structural Biology of Gaucher Disease: Acid-β-Glucosidase and Saposin C. Enzyme Research 973231, 2011). Its activity can be measured using any commercially available kits, such as the Glucosylceramidase/GBA Assay Kit (colorimetric) (Cat. No. KA1611), marketed by Novus Biologicals, LLC (Centennial, Colo.). Thus it is well within the skill of one of ordinary skill in the art to make conservative amino acid changes without significantly/adversely affecting the catalytic activity of β-glucocerebrosidase.

A polynucleotide sequence encoding the above β-glucocerebrosidase fusion is provided.

(SEQ ID NO: 9) [symbol]
ATGGACCCGCCACG
GCCAGCTCTGCTCGCACTTTTGGCTCTTCCGGCGTTGCTGCTGTTGCTTC
TGGCTGGCGCGAGAGCTGCGCGGCCGTGTATACCGAAGTCTTTCGGGTAC
TCCTCCGTCGTATGCGTCTGCAACGCTACTTACTGTGACTCTTTCGATCC
TCCGACGTTTCCAGCTCTCGGGACTTTCTCACGATATGAGTCCACTAGGT
CTGGTCGAAGAATGGAACTTAGTATGGGTCCAATACAAGCGAACCACACC
GGGACTGGACTGCTCCTTACGCTTCAGCCGGAACAGAAATTTCAGAAGGT
TAAAGGGTTTGGTGGAGCGATGACTGACGCTGCAGCACTGAATATCCTCG
CATTGTCTCCCCCCGCGCAAAATCTGTTGCTTAAGTCTTATTTTTCTGAG
GAGGGCATAGGTTACAACATAATTCGGGTGCCAATGGCTAGCTGCGACTT
TAGCATTCGCACATACACGTATGCAGATACTCCCGATGACTTTCAACTGC
ACAACTTCTCTTTGCCTGAGGAAGATACGAAACTCAAAATACCACTCATA
CATAGGGCATTGCAGTTGGCTCAAAGGCCTGTTAGTTTGCTCGCCAGTCC
CTGGACCAGCCCGACATGGCTGAAGACAAATGGTGCTGTAAATGGCAAAG
GCTCTCTCAAGGGGCAGCCAGGTGATATTTATCATCAAACATGGGCACGG
TATTTTGTTAAGTTTCTCGATGCTTATGCAGAACACAAACTGCAATTTTG
GGCTGTAACTGCGGAAAATGAACCCAGCGCGGGGCTCCTGTCCGGCTACC
CGTTCCAATGCCTGGGATTCACGCCAGAACATCAACGGGACTTCATCGCG
CGCGATTTGGGTCCAACGCTGGCCAACTCAACACATCACAACGTCCGACT
GTTGATGCTTGACGACCAACGCCTTCTCTTGCCCCATTGGGCCAAAGTCG
TTCTCACAGATCCTGAGGCAGCAAAGTACGTTCATGGTATTGCTGTGCAT
TGGTACCTCGACTTTTTGGCGCCGGCGAAGGCTACCCTGGGGGAAACTCA
CCGGCTGTTCCCTAATACCATGCTGTTTGCGTCCGAAGCGTGTGTGGGTT
CCAAGTTTTGGGAACAAAGTGTCCGGCTCGGAAGCTGGGACCGGGGCATG
CAGTATTCTCACTCTATTATCACCAACCTTTTGTATCACGTGGTGGGCTG
GACGGACTGGAACTTGGCACTGAATCCAGAGGGTGGCCCTAATTGGGTGC
GCAACTTTGTAGATAGTCCGATTATCGTTGACATCACTAAGGACACCTTC
TATAAGCAGCCTATGTTTTACCACCTCGGGCACTTCAGCAAATTCATCCC
AGAGGGGTCCCAGAGGGTAGGACTGGTCGCGTCTCAGAAGAACGATCTTG
ACGCGGTGGCCTTGATGCACCCAGATGGGAGCGCAGTCGTGGTTGTGCTT
AATAGAAGTAGTAAAGACGTCCCCCTCACAATAAAAGACCCGGCTGTGGG
ATTCCTGGAAACCATCTCCCCCGGGTATAGTATACATACTTACCTCTGGA
GACGGCAGCACCACCACCATCATCACTAG [symbol]
GGATCCTCTAGAGTCGACCTGCAG
AA

The double underlined sequence at the 5′-end and the bold italic sequence at the 3′-end represent end tag sequences that can be used for manufacturing/amplification of the coding sequences in the middle.

In one embodiment, the GOI is iduronate-2 sulfatase, and the disease or condition treatable by the subject AAV vector/virus is Hunter Syndrome (mucopolysaccharidosis type II, or MPS II).

Hunter Syndrome is a rare genetic disorder also falling within the group of lysosomal storage diseases. Hunter Syndrome is caused by a deficiency of the lysosomal enzyme iduronate-2-sulfatase (I2S), leading to accumulation of glycosaminoglycans (GAGs, or mucopolysaccharides) such as heparan sulfate and dermatan sulfate in all body tissues, including many organs such as the skeleton, heart, and respiratory system. Hunter syndrome is the only MPS syndrome to exhibit X-linked recessive inheritance.

Idursulfase, a purified form of the missing lysosomal enzyme, underwent clinical trial in 2006 and was subsequently approved by the US FDA as an enzyme replacement treatment for Hunter syndrome. It costs well over $500,000 a year per patient for life time treatment. Idursulfase beta, another enzyme replacement treatment, was approved in Korea. Recent advances in enzyme replacement therapy (ERT) with idursulfase have been proven to improve many signs and symptoms of MPS II, especially if started early in the disease.

The composition and methods of the invention can be used to produce iduronate-2 sulfatase expressed permanently from AAV vectors stably maintained with the host's genome as extra chromosomal material, and secreted into a patient's blood stream, thus effectively treating Hunter Syndrome.

A representative iduronate-2 sulfatase fusion protein that can be encoded by the AAV vector of the invention is described below.

(SEQ ID NO: 10) [symbol]
SETQANSTTDALNVLLIIVDDLR
PSLGCYGDKLVRSPNIDQLASHSLLFQNAFAQQAVCAPSRVSFLTGRRPD
TTRLYDFNSYWRVHAGNFSTIPQYFKENGYVTMSVGKVFHPGISSNHTDD
SPYSWSFPPYHPSSEKYENTKTCRGPDGELHANLLCPVDVLDVPEGTLPD
KQSTEQAIQLLEKMKTSASPFFLAVGYHKPHIPFRYPKEFQKLYPLENIT
LAPDPEVPDGLPPVAYNPWMDIRQREDVQALNISVPYGPIPVDFQRKIRQ
SYFASVSYLDTQVGRLLSALDDLQLANSTIIAFTSDHGWALGEHGEWAKY
SNFDVATHVPLIFYVPGRTASLPEAGEKLFPYLDPFDSASQLMEPGRQSM
DLVELVSLFPTLAGLAGLQVPPRCPVPSFHVELCREGKNLLKHFRFRDLE
EDPYLPGNPRELIAYSQYPRPSDIPQWNSDKPSLKDIKIMGYSIRTIDYR
YTVWVGFNPDEFLANFSDIHAGELYFVDSDPLQDHNMYNDSQGGDLFQLL
MP

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Crystal structure of the human iduronate-2 sulfatase with a covalently bound sulfate ion in the active site has been resolved (Demydchuk et al., Nat Commun. 8: 15786, 2017; Published online doi: 10.1038/ncomms15786). This structure provides essential insight into multiple mechanisms by which pathogenic mutations interfere with enzyme function, and a compelling explanation for severe Hunter syndrome phenotypes. Thus it is well within the skill of one of ordinary skill in the art to make conservative amino acid changes without significantly/adversely affecting the catalytic activity of iduronate-2 sulfatase.

A polynucleotide sequence encoding the above iduronate-2 sulfatase fusion is provided.

(SEQ ID NO: 11) [symbol]
ATGGACCCACCTCG
CCCGGCCCTTCTCGCGTTGCTTGCTCTCCCTGCCTTGCTTCTGTTGTTGC
TTGCCGGAGCACGCGCCAGTGAGACCCAGGCCAATAGTACTACGGACGCG
CTCAACGTTCTCCTCATAATAGTGGATGATCTGCGGCCATCATTGGGGTG
CTACGGCGACAAACTTGTTAGGTCTCCTAATATTGACCAATTGGCTAGTC
ATTCTCTTCTTTTCCAGAACGCATTCGCGCAGCAAGCGGTTTGCGCTCCC
TCTAGGGTTAGTTTCCTCACTGGACGAAGACCCGACACGACAAGACTTTA
TGATTTCAATTCATACTGGCGAGTTCACGCCGGCAATTTTTCAACCATCC
CGCAGTACTTTAAGGAAAACGGCTACGTGACAATGAGCGTTGGGAAGGTA
TTCCATCCTGGGATTTCCTCCAATCACACGGACGACAGTCCTTACAGTTG
GAGTTTCCCACCCTATCACCCCTCATCCGAAAAATATGAGAATACTAAAA
CTTGCCGAGGTCCTGACGGTGAGCTTCATGCGAATCTTTTGTGTCCGGTC
GATGTTCTGGATGTACCGGAAGGAACGCTCCCCGATAAGCAAAGTACTGA
GCAGGCGATTCAACTTTTGGAGAAAATGAAAACAAGCGCATCTCCCTTTT
TCTTGGCTGTCGGCTATCATAAGCCACACATCCCATTCCGCTATCCGAAG
GAGTTTCAGAAGCTCTATCCCCTTGAGAATATAACACTGGCTCCAGACCC
CGAAGTGCCAGACGGACTCCCTCCTGTCGCTTACAATCCTTGGATGGACA
TCAGACAGAGAGAGGATGTACAGGCACTTAATATAAGCGTCCCCTACGGA
CCTATACCAGTGGATTTCCAGCGCAAAATCCGGCAAAGTTATTTTGCGAG
TGTGAGCTATCTGGATACCCAGGTCGGTCGCTTGTTGTCCGCCCTCGATG
ACCTGCAGCTCGCCAACAGCACGATCATTGCATTCACTTCTGATCACGGG
TGGGCTCTGGGTGAGCATGGGGAGTGGGCGAAGTATAGCAATTTCGACGT
TGCAACGCACGTTCCACTTATATTTTATGTACCTGGCCGAACAGCATCCC
TTCCCGAAGCTGGCGAGAAGTTGTTTCCTTACTTGGACCCCTTCGATAGC
GCGAGTCAGCTGATGGAACCTGGCAGACAAAGTATGGACCTTGTTGAACT
GGTCTCTCTGTTTCCAACTTTGGCAGGGCTGGCTGGCCTGCAAGTACCAC
CGAGATGCCCAGTCCCGTCATTCCACGTGGAGCTCTGCCGAGAAGGTAAG
AACCTTTTGAAGCACTTCAGGTTCCGGGACCTCGAGGAAGATCCGTATTT
GCCCGGTAACCCGCGAGAGCTTATAGCATACAGCCAGTACCCGCGCCCAA
GCGACATACCTCAGTGGAATAGCGACAAACCATCCCTGAAGGACATTAAA
ATAATGGGGTATAGCATCAGGACGATTGATTATAGGTACACCGTCTGGGT
TGGGTTTAACCCAGACGAGTTCCTGGCAAACTTTTCTGATATCCACGCAG
GGGAACTCTACTTTGTGGATTCCGACCCACTGCAAGATCACAATATGTAT
AATGATTCTCAGGGCGGAGACCTCTTCCAGCTGCTCATGCCGCACCATCA
TCATCATCACTAG

[symbol]

[symbol]

The double underlined sequence at the 5′-end and the bold italic sequence at the 3′-end represent end tag sequences that can be used for manufacturing/amplification of the coding sequences in the middle.

In one embodiment, the GOI is TNSALP (tissue non-specific alkaline phosphatase), and the disease or condition treatable by the subject AAV vector/virus is hypophosphatasia (also called deficiency of alkaline phosphatase, phosphoethanolaminuria, or Rathbun's Syndrome), including perinatal hypophosphatasia, infantile hypophosphatasia, childhood hypophosphatasia, and adult hypophosphatasia. The disease was discovered initially in 1936, but was fully named and documented by a Canadian Pediatrician named John Campbell Rathbun while examining and treating a baby boy with very low levels of alkaline phosphatase in 1948. The genetic basis of the disease was mapped out only some 40 years later.

Hypophosphatasia is a rare, and sometimes fatal, metabolic bone disease. Genetic inheritance is autosomal recessive for the perinatal and infantile forms but either autosomal recessive or autosomal dominant in the milder forms. The pathognomonic finding is subnormal serum activity of the TNSALP enzyme, which is caused by one of 200 genetic mutations identified to date, in the gene encoding TNSALP. TNSALP is an enzyme that is tethered to the outer surface of osteoblasts and chondrocytes. It hydrolyzes several substances, including inorganic pyrophosphate (PPi) and pyridoxal 5′-phosphate (PLP), a major form of vitamin B₆.

TNSALP is an alkaline phosphatase (ALP). Unlike bacterial ALPs, mammalian ALPs are modified with N-glycan and anchored to the cell membrane via glycosylphosphatidylinositol (GPI). In humans, four ALP isozymes are encoded by separate genes: tissue-nonspecific ALP (TNSALP), intestinal ALP, placental ALP and germ cell (placental-like) ALP. TNSALP is expressed in a multitude of tissues, and the ALPL gene encoding TNSALP is localized on human chromosome 1 (1p36-p34). The three other ALPs are named as tissue-specific enzymes after their restricted expression in the body and their genes are clustered on human chromosome 2, bands q34-q37. Tissue-specific ALPs are closely related to one another with homologies of more than 90%, whereas the homology between placental ALP and TNSALP is 74%.

TNSALP deficiency in osteoblasts and chondrocytes impairs bone mineralization, leading to rickets or osteomalacia. Clinical symptoms of this disease are heterogeneous, ranging from the rapidly fatal, perinatal variant, with profound skeletal hypomineralization and respiratory compromise, to a milder, progressive osteomalacia later in life. On the other hand, tissue-specific ALPs have not yet been related to any genetic disorder.

An ERT for the treatment of hypophosphatasia (e.g., patients with perinatal/infantile- and juvenile-onset hypophosphatasia) was approved by the US FDA in October 2015, as asfotase alfa (Strensiq). Asfotase alfa is a recombinant glycoprotein that contains the catalytic domain of TNSALP. The peptide part of the glycoprotein asfotase alfa consists of two identical chains of 726 amino acids each, containing (1) the catalytic domain of TNSALP, (2) the Fc region of human immunoglobulin G1, and (3) a sequence of ten L-aspartate residues at the carboxy terminus. The two chains are linked by two disulfide bridges. Each chain also contains four internal disulfide bridges. It is introduced into the patient by s.c. injection.

The composition and methods of the invention can be used to produce TNSALP or functional equivalent of asfotase alfa expressed permanently from AAV vectors stably maintained with the host's genome as extra chromosomal material, and secreted into a patient's blood stream, thus effectively treating various forms of hypophosphatasia.

A representative TNSALP fusion protein that can be encoded by the AAV vector of the invention is described below.

(SEQ ID NO: 12) [symbol]
LVPEKEKDPKYWRDQAQETLKYA
LELQKLNTNVAKNVIMFLGDGMGVSTVTAARILKGQLHHNPGEETRLEMD
KFPFVALSKTYNTNAQVPDSAGTATAYLCGVKANEGTVGVSAATERSRCN
TTQGNEVTSILRWAKDAGKSVGIVTTTRVNHATPSAAYAHSADRDWYSDN
EMPPEALSQGCKDIAYQLMHNIRDIDVIMGGGRKYMYPKNKTDVEYESDE
KARGTRLDGLDLVDTWKSFKPRYKHSHFIWNRTELLTLDPHNVDYLLGLF
EPGDMQYELNRNNVTDPSLSEMVVVAIQILRKNPKGFFLLVEGGRIDHGH
HEGKAKQALHEAVEMDRAIGQAGSLTSSEDTLTVVTADHSHVFTFGGYTP
RGNSIFGLAPMLSDTDKKPFTAILYGNGPGYKVVGGERENVSMVDYAHNN
YQAQSAVPLRHETHGGEDVAVFSKGPMAHLLHGVHEQNYVPHVMAYAACI
GANLGHCAPASS

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of TNSALP—an alkaline phosphatasesha—long been well-characterized. Alkaline phosphatases (ALP; EC3.1.3.1) are widely distributed in many organisms from bacteria to humans. The catalytic mechanism of ALPs has been well conserved in evolution. Most ALPs are a homodimer and each subunit contains an active center serine residue surrounded by two zinc ions and one magnesium ion (Millan, Mammalian Alkaline Phosphatases: From Biology to Applications in Medicine and Biotechnology. Print ISBN:9783527310791|Online ISBN:9783527608065 POI:10.1002/3527608060, 2006, Wiley-VCH Verlag GmbH & Co. KGaA). ALPs catalyze the hydrolysis of phosphomonoester compounds. ALP activity can be measured using any art recognized method (such as that described in Fukushi-Irie et al., Biochem J 348, 633-642, 2000), or using any commercially available kits, such as the Alkaline Phosphatase Assay Kit (Colorimetric) (Cat. No. ab83369), marketed by Abcam (Cambridge, UK). Thus it is well within the skill of one of ordinary skill in the art to make conservative amino acid changes without significantly/adversely affecting the catalytic activity of TNSALP.

A polynucleotide sequence encoding the above TNSALP fusion is provided.

(SEQ ID NO: 13) [symbol]
ATGGACCCGCCGAG
GCCCGCACTGCTGGCACTTCTCGCTCTTCCCGCGCTCCTGTTGCTCTTGC
TGGCCGGAGCACGCGCACTCGTACCTGAAAAGGAAAAAGATCCAAAATAC
TGGAGAGACCAAGCACAGGAAACCTTGAAGTATGCATTGGAGCTTCAGAA
GCTTAACACAAACGTAGCAAAGAACGTTATAATGTTCCTTGGCGACGGTA
TGGGCGTTAGCACGGTTACGGCAGCAAGAATTTTGAAGGGGCAGTTGCAC
CACAATCCCGGGGAGGAGACAAGACTCGAAATGGACAAATTTCCGTTCGT
AGCACTGTCCAAGACTTACAACACTAATGCACAGGTTCCCGATTCTGCGG
GCACTGCAACAGCTTATCTGTGCGGCGTCAAAGCAAACGAGGGAACTGTG
GGAGTGTCCGCTGCCACTGAGCGCAGTCGCTGCAACACAACACAAGGTAA
CGAGGTGACAAGCATTCTGAGATGGGCTAAGGATGCGGGCAAGTCCGTTG
GAATAGTAACCACTACCCGAGTGAACCACGCGACTCCGTCAGCGGCGTAT
GCCCATAGCGCCGACAGAGACTGGTACTCAGACAATGAAATGCCTCCCGA
AGCGTTGAGTCAAGGTTGCAAGGACATTGCGTACCAGCTGATGCACAATA
TTAGAGATATCGACGTCATAATGGGCGGAGGACGGAAGTATATGTATCCA
AAGAATAAGACGGATGTGGAGTATGAGTCAGACGAGAAAGCCAGAGGCAC
GCGGCTTGATGGTCTGGATCTTGTGGATACATGGAAATCATTTAAGCCTC
GATATAAACATAGTCATTTTATTTGGAACCGGACTGAACTCCTCACCCTC
GACCCCCATAATGTTGACTACTTGCTTGGTCTTTTCGAGCCTGGTGATAT
GCAATACGAGCTCAACCGCAATAACGTTACTGACCCCAGCCTCAGCGAAA
TGGTCGTTGTTGCTATCCAGATACTGAGAAAAAACCCTAAGGGCTTTTTT
CTCTTGGTAGAGGGCGGAAGGATAGATCACGGTCATCATGAAGGAAAAGC
CAAACAAGCCCTGCACGAGGCCGTCGAGATGGATCGAGCAATTGGACAGG
CCGGTAGTCTGACGTCCAGCGAGGATACTCTGACTGTAGTTACCGCTGAC
CACTCCCACGTTTTTACCTTCGGTGGTTATACACCTAGAGGCAATTCCAT
TTTTGGCTTGGCTCCCATGCTTTCAGACACTGACAAGAAGCCATTTACTG
CTATCCTCTACGGGAACGGGCCAGGCTACAAGGTAGTAGGGGGAGAAAGA
GAGAACGTGTCAATGGTCGATTACGCGCACAACAATTACCAGGCTCAGTC
AGCAGTACCCCTCCGACACGAGACGCACGGTGGCGAGGATGTAGCCGTTT
TCAGCAAAGGGCCGATGGCCCATCTTCTTCATGGAGTACATGAACAGAAC
TACGTACCACACGTAATGGCGTATGCGGCGTGCATTGGCGCGAATCTGGG
CCACTGCGCACCGGCGTCTTCACATCATCACCACCACCACTAG

[symbol]

[symbol]

The double underlined sequence at the 5′-end and the bold italic sequence at the 3′-end represent end tag sequences that can be used for manufacturing/amplification of the coding sequences in the middle.

In one embodiment, the GOI is Lysosomal Acid Lipase (LAL), and the disease or condition treatable by the subject AAV vector/virus is LAL Deficiency (LAL-D).

LAL-D is an ultra-rare, chronic, and progressive metabolic autosomal recessive genetic disease associated with significant morbidity and premature mortality. In LAL-D patients, genetic mutations result in decreased activity of the LAL enzyme, leading to marked accumulation of cholesteryl esters and triglycerides in vital organs, blood vessels, and other tissues, which in turn lead to progressive and multi-organ damage, including fibrosis, cirrhosis, liver failure, accelerated atherosclerosis, cardiovascular disease, and other devastating consequences.

An ERT for LAL-D was first approved in the US as Sebelipase Alfa (KANUMA™), which reduces substrate accumulation in the lysosomes of cells throughout the body. Clinical data shows that Sebelipase Alfa (KANUMA™) improved survival in infants with LAL-D and led to significant reductions in ALT and liver fat content, as well as significant improvements in lipid parameters, in children and adults with LAL-D. Sebelipase Alfa (KANUMA™) is a recombinant form of LAL, and is administered to patients through IV infusion. According to an estimate by a Barclays analyst in 2015, the drug would be priced at about US $375,000 per year.

The composition and methods of the invention can be used to produce LAL or sebelipase alfa expressed permanently from AAV vectors stably maintained with the host's genome as extra chromosomal material, and secreted into a patient's blood stream, thus effectively treating LAL-D.

A representative lysosomal acid lipase fusion protein that can be encoded by the AAV vector of the invention is described below.

(SEQ ID NO: 14) [symbol]
SGGKLTAVDPETNMNVSEIISYW
GFPSEEYLVETEDGYILCLNRIPHGRKNHSDKGPKPVVFLQHGLLADSSN
WVTNLANSSLGFILADAGFDVWMGNSRGNTWSRKHKTLSVSQDEFWAFSY
DEMAKYDLPASINFILNKTGQEQVYYVGHSQGTTIGFIAFSQIPELAKRI
KMFFALGPVASVAFCTSPMAKLGRLPDHLIKDLFGDKEFLPQSAFLKWLG
THVCTHVILKELCGNLCFLLCGFNERNLNMSRVDVYTTHSPAGTSVQNML
HWSQAVKFQKFQAFDWGSSAKNYFHYNQSYPPTYNVKDMLVPTAVWSGGH
DWLADVYDVNILLTQITNLVFHESIPEWEHLDFIWGLDAPWRLYNKIINL
MRKYQ

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of LAL has long been well-characterized as part of the diagnosis of LAL-D. LAL activity can be measured in patient leukocytes using flurogenic 4-MU oleate substrate and LAL inhibitor lalistat 2 for subtracting the non-LAL lipases. Thus it is well within the skill of one of ordinary skill in the art to make conservative amino acid changes without significantly/adversely affecting the catalytic activity of LAL.

A polynucleotide sequence encoding the above LAL fusion is provided.

(SEQ ID NO: 15) [symbol]
ATGGACCCCCCACG
ACCTGCTCTTCTTGCTTTGCTCGCTTTGCCCGCATTGCTCCTGCTCCTTC
TGGCGGGCGCCCGGGCCTCTGGCGGCAAATTGACGGCGGTCGATCCAGAG
ACGAACATGAACGTATCCGAAATAATCTCCTACTGGGGGTTTCCTTCTGA
AGAATATCTTGTTGAGACCGAAGATGGTTATATACTCTGCCTCAACCGAA
TCCCACATGGGAGGAAGAATCACTCCGATAAAGGCCCAAAACCTGTCGTC
TTCTTGCAGCATGGTTTGCTGGCGGATAGCTCAAACTGGGTTACCAACCT
TGCCAACTCCAGTCTGGGGTTCATACTCGCGGACGCGGGCTTCGACGTCT
GGATGGGGAACAGCCGGGGTAATACTTGGAGCCGAAAACATAAGACTCTT
TCAGTCTCCCAGGACGAGTTCTGGGCGTTCTCATATGATGAAATGGCTAA
ATACGATCTCCCAGCATCAATCAATTTCATTCTCAACAAAACAGGTCAAG
AGCAGGTTTACTACGTAGGGCATTCCCAGGGCACCACCATAGGTTTCATT
GCCTTTTCTCAGATCCCCGAACTGGCAAAGCGCATAAAAATGTTTTTCGC
CCTTGGCCCTGTGGCATCCGTAGCGTTCTGTACTAGCCCTATGGCAAAGT
TGGGTCGCCTGCCTGATCACCTTATCAAAGATCTGTTTGGAGACAAGGAG
TTTCTTCCACAATCCGCTTTCTTGAAGTGGCTCGGCACCCACGTTTGTAC
ACACGTGATCCTTAAAGAGCTTTGTGGCAATTTGTGTTTCCTGCTCTGCG
GTTTTAACGAGCGCAACCTCAACATGAGCCGAGTAGATGTGTATACCACC
CACAGCCCCGCCGGAACCTCAGTACAAAACATGCTTCATTGGTCACAGGC
AGTGAAATTCCAAAAATTCCAAGCATTCGATTGGGGCAGTAGCGCTAAGA
ATTATTTCCACTACAATCAGTCATACCCGCCGACGTATAATGTGAAGGAT
ATGCTGGTGCCGACCGCAGTATGGTCCGGCGGGCACGATTGGCTCGCCGA
CGTCTACGATGTAAACATTCTGCTCACGCAAATCACGAACCTTGTCTTCC
ACGAAAGCATCCCAGAATGGGAGCATCTTGATTTTATCTGGGGTTTGGAT
GCCCCCTGGAGGTTGTATAATAAAATTATCAATTTGATGAGGAAGTACCA
GCACCACCACCACCATCATTAG

[symbol]

The double underlined sequence at the 5′-end and the bold italic sequence at the 3′-end represent end tag sequences that can be used for manufacturing/amplification of the coding sequences in the middle.

In one embodiment, the GOI is alpha-L-iduronidase (IDUA), and the disease or condition treatable by the subject AAV vector/virus is mucopolysaccharidosis (MPS).

Mucopolysaccharidosis is a group of inherited conditions caused by defective activity of the lysosomal enzymes that degrade mucopolysaccharides (glycosaminoglycan, or GAG, chains). This results in abnormal accumulation of partially degraded GAGs, dermatan sulfate and heparan sulfate throughout the body and leads to widespread cellular, tissue and organ dysfunction. Seven distinct forms and numerous subtypes of MPS have been identified. The underlying genetic cause varies by form. More than 100 mutations in the IDUA gene have been found to cause MPS type I. In general, most affected people appear healthy at birth and experience a period of normal development, followed by a decline in physical and/or mental function. As the condition progresses, it may affect appearance, physical abilities, organ functions, and, in most cases, cognitive development.

An ERT for IDUA was first approved in the US as Aldurazyme (laronidase), which catalyzes the hydrolysis of terminal alpha-L-iduronic acid residues of dermatan sulfate and heparan sulfate. Aldurazyme has been indicated for patients with Hurler and Hurler-Scheie forms of MPS type I. Clinical data shows that patients treated with Aldurazyme showed improvement in forced vital capacity and distance walked in 6 minutes compared to placebo-treated patients. Aldurazyme is produced by recombinant DNA technology and is administered to patients through IV infusion.

The composition and methods of the invention can be used to produce IDUA or Aldurazyme expressed permanently from AAV vectors stably maintained with the host's genome as extra chromosomal material, and secreted into a patient's blood stream, thus effectively treating MPS.

A representative IDUA fusion protein that can be encoded by the AAV vector of the invention is described below.

(SEQ ID NO: 16) [symbol]
AAPPVAPAEAPHLVHVDAARALW
PLRRFWRSTGFCPPLPHSQADQYVLSWDQQLNLAYVGAVPHRGIKQVRTH
WLLELVTTRGSTGRGLSYNFTHLDGYLDLLRENQLLPGFELMGSASGHFT
DFEDKQQVFEWKDLVSSLARRYIGRYGLAHVSKWNFETWNEPDHHDFDNV
SMTMQGFLNYYDACSEGLRAASPALRLGGPGDSFHTPPRSPLSWGLLRHC
HDGTNFFTGEAGVRLDYISLHRKGARSSISILEQEKVVAQQIRQLFPKFA
DTPIYNDEADPLVGWSLPQPWRADVTYAAMVVKVIAQHQNLLLANTTSAF
PYALLSNDNAFLSYHPHPFAQRTLTARFQVNNTRPPHVQLLRKPVLTAMG
LLALLDEEQLWAEVSQAGTVLDSNHTVGVLASAHRPQGPADAWRAAVLIY
ASDDTRAHPNRSVAVTLRLRGVPPGPGLVYVTRYLDNGLCSPDGEWRRLG
RPVFPTAEQFRRMRAAEDPVAAAPRPLPAGGRLTLRPALRLPSLLLVHVC
ARPEKPPGQVTRLRALPLTQGQLVLVWSDEHVGSKCLWTYEIQFSQDGKA
YTPVSRKPSTFNLFVFSPDTGAVSGSYRVRALDYWARPGPFSDPVPYLEV
PVPRGPPSPGNP

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of IDUA has long been well-characterized as part of the diagnosis of MPS type I. IDUA activity can be measured by its ability to cleave a flurogenic substrate such as 4-methylumbelliferyl alpha-L-iduronide. Thus it is well within the skill of one of ordinary skill in the art to make conservative amino acid changes without significantly/adversely affecting the catalytic activity of IDUA.

A polynucleotide sequence encoding the above IDUA fusion is provided.

(SEQ ID NO: 17) [symbol]
ATGGACCCCCCACG
ACCTGCTCTTCTTGCTTTGCTCGCTTTGCCCGCATTGCTCCTGCTCCTTC
TGGCGGGCGCCCGGGCCGCCGCACCTCCCGTTGCCCCTGCGGAGGCACCA
CATCTCGTTCATGTTGACGCTGCGAGGGCTTTGTGGCCACTTAGGCGATT
CTGGAGAAGTACGGGCTTCTGTCCACCGCTGCCGCACTCACAGGCAGACC
AATATGTCTTGAGCTGGGATCAGCAACTCAACTTGGCGTATGTGGGAGCT
GTCCCGCATCGCGGTATTAAGCAAGTGCGAACGCATTGGTTGCTTGAGTT
GGTAACCACGAGAGGTTCCACTGGGCGAGGGCTCTCCTATAACTTCACAC
ACCTCGATGGGTATCTGGACCTGCTGCGAGAAAATCAACTCCTTCCCGGG
TTCGAACTGATGGGCTCCGCATCTGGCCACTTCACGGACTTCGAAGATAA
GCAGCAGGTCTTCGAATGGAAAGATCTGGTGTCAAGTCTGGCTCGCAGGT
ATATTGGACGATACGGTCTGGCCCACGTCAGTAAGTGGAATTTTGAAACG
TGGAACGAACCAGACCACCATGACTTTGATAACGTATCTATGACTATGCA
GGGATTTCTCAATTACTATGACGCTTGCTCTGAAGGACTCCGGGCTGCAA
GTCCAGCTCTCCGCTTGGGCGGGCCTGGGGATAGTTTCCATACACCTCCC
CGATCCCCGTTGAGTTGGGGGCTCCTCCGACATTGTCACGACGGGACGAA
CTTTTTCACCGGTGAAGCGGGAGTTCGGCTCGATTACATCTCTCTGCATC
GGAAGGGCGCAAGAAGCTCCATAAGTATCTTGGAGCAAGAGAAAGTCGTG
GCGCAGCAGATCAGACAGCTCTTCCCGAAGTTTGCGGATACACCTATATA
CAATGATGAGGCTGACCCACTGGTCGGCTGGAGCCTGCCCCAGCCGTGGA
GGGCTGACGTAACATACGCCGCGATGGTGGTTAAGGTAATAGCCCAGCAC
CAGAATCTTCTGTTGGCAAATACCACGAGTGCATTCCCTTATGCTCTGTT
GTCAAATGATAATGCCTTCTTGTCTTACCACCCGCATCCTTTCGCTCAAC
GCACGCTGACCGCCCGCTTTCAAGTAAATAACACTCGCCCACCCCACGTG
CAGCTCCTGAGGAAGCCCGTTCTGACTGCTATGGGGCTCCTCGCGCTGCT
GGATGAGGAGCAGTTGTGGGCTGAGGTATCTCAAGCCGGGACTGTATTGG
ACTCCAATCACACTGTGGGGGTTCTTGCTAGCGCGCACCGCCCTCAAGGA
CCTGCCGACGCCTGGCGCGCTGCAGTGTTGATATATGCCAGTGATGATAC
GCGAGCCCACCCGAATAGGTCTGTTGCCGTGACGTTGCGCCTCAGAGGCG
TTCCCCCTGGTCCAGGACTCGTCTATGTTACTCGATATTTGGATAACGGC
CTCTGTTCCCCTGACGGTGAATGGCGGAGATTGGGGAGACCTGTGTTTCC
GACAGCGGAGCAGTTCCGGCGCATGCGAGCGGCAGAAGACCCTGTTGCAG
CTGCTCCACGACCGCTCCCTGCAGGCGGCAGGCTTACTTTGAGACCAGCT
TTGAGGTTGCCGAGCCTCCTGTTGGTCCATGTATGCGCGCGGCCAGAGAA
GCCACCCGGGCAAGTAACCCGGCTTCGCGCCCTCCCGCTCACCCAAGGCC
AGCTCGTGCTTGTATGGAGCGACGAGCATGTTGGATCTAAGTGTTTGTGG
ACTTACGAGATACAATTTAGCCAGGATGGAAAGGCGTATACCCCTGTGAG
TCGGAAGCCTTCTACATTCAACCTCTTCGTGTTCAGCCCCGACACAGGTG
CTGTTAGCGGGTCCTATCGGGTGCGGGCGCTGGATTACTGGGCACGGCCC
GGGCCGTTTTCCGACCCTGTTCCCTACCTGGAAGTTCCCGTTCCGCGAGG
CCCACCTAGCCCTGGGAATCCACACCACCACCACCATCATTAG

[symbol]

[symbol]

The double underlined sequence at the 5′-end and the bold italic sequence at the 3′-end represent end tag sequences that can be used for manufacturing/amplification of the coding sequences in the middle.

In one embodiment, the GOI is arylsulfatase B (ARSB), and the disease or condition treatable by the subject AAV vector/virus is maroteux lamy.

Maroteux lamy syndrome (also known as mucopolysaccharidosis type VI, MPS IV) is a rare genetic disorder characterized by complete or partial lack of activity of the enzyme ARSB. More than 100 mutations in the ARSB gene have been found to cause maroteux lamy/MPS type VI. The lack of ARSB activity leads to the accumulation of GAGS in lysosomes. Patients with maroteux lamy syndrome generally do not display any features of the condition at birth. They often begin to show signs and symptoms during early childhood. The features of maroteux lamy syndrome include a large head, a buildup of fluid in the brain, distinctive-looking facial features that are described as “coarse,” and a large tongue. Affected individuals also frequently develop heart valve abnormalities, an enlarged liver and spleen, and a soft out-pouching around the belly-button or lower abdomen. The airway may become narrow in some patients, leading to frequent upper respiratory infections and short pauses in breathing during sleep (sleep apnea). The clear covering of the eye (cornea) typically becomes cloudy, which can cause significant vision loss. Patients with maroteux lamy syndrome may also have recurrent ear infections and hearing loss. Unlike other types of mucopolysaccharidosis, maroteux lamy syndrome does not affect intelligence. Maroteux lamy syndrome also causes various skeletal abnormalities, including short stature and joint deformities that affect mobility.

An ERT for ARSB was first approved in the US as Naglazyme (galsulfase), which catalyzes the hydrolysis of the sulfate ester from terminal N-acetylgalactosamine 4-sulfate residues of chondroitin 4-sulfate and dermatan sulfate. Naglazyme has been shown to improve walking and stair-climbing capacity of maroteux lamy syndrome/MPS type VI patients. Naglzyme is produced by recombinant DNA technology and is administered to patients through IV infusion.

The composition and methods of the invention can be used to produce ARSB or Naglazyme expressed permanently from AAV vectors stably maintained with the host's genome as extra chromosomal material, and secreted into a patient's blood stream, thus effectively treating maroteux lamy syndrome/MPS type VI.

A representative ARSB fusion protein that can be encoded by the AAV vector of the invention is described below.

(SEQ ID NO: 18) [symbol]
SGAGASRPPHLVFLLADDLGWND
VGFHGSRIRTPHLDALAAGGVLLDNYYTQPLCTPSRSQLLTGRYQIRTGL
QHQIIWPCQPSCVPLDEKLLPQLLKEAGYTTHMVGKWHLGMYRKECLPTR
RGFDTYFGYLLGSEDYYSHERCTLIDALNVTRCALDFRDGEEVATGYKNM
YSTNIFTKRAIALITNHPPEKPLFLYLALQSVHEPLQVPEEYLKPYDFIQ
DKNRHHYAGMVSLMDEAVGNVTAALKSSGLWNNTVFIFSTDNGGQTLAGG
NNWPLRGRKWSLWEGGVRGVGFVASPLLKQKGVKNRELIHISDWLPTLVK
LARGHTNGTKPLDGFDVWKTISEGSPSPRIELLHNIDPNFVDSSPCPRNS
MAPAKDDSSLPEYSAFNTSVHAAIRHGNWKLLTGYPGCGYWFPPPSQYNV
SEIPSSDPPTKTLWLFDIDRDPEERHDLSREYPHIVTKLLSRLQFYHKHS
VPVYFPAQDPRCDPKATGVWGPWM

[symbol]

Bold and double underlined sequence at the N-terminus is the ApoB signal sequence (which may be replaced by any one of the secretary signal peptide of the invention as described herein), and the bold and italic sequence at the C-terminus is the optional 6 His-tag that is useful for verifying expression but is not needed for therapeutic effect (and thus can be omitted in a clinical version of the sequence).

Conservative changes to the sequence above (e.g., 1, 2, 3, 4, 5, 6, 7, 8, 9, 10, 11, 12, 13, 14, 15, 16, 17, 18, 19, or 20 conservative residue changes, or up to 1%, 2%, 3%, 4%, 5%, 6%, 7%, 8%, 9%, or 10% conservative residue changes) that result in a functional equivalent version of the protein (e.g., that retains at least 70%, 75%, 80%, 85%, 90%, 95%, 97%, 99% of the wild-type enzyme activity) are also within the scope of the invention.

Catalytic activity of ARSB has long been well-characterized as part of the diagnosis of maroteux lamy syndrome. ARSB activity can be measured by its ability to hydrolyze substrates such as 4-nitrocatechol sulfate. Thus it is well within the skill of one of ordinary skill in the art to make conservative amino acid changes without significantly/adversely affecting the catalytic activity of ARSB.

A polynucleotide sequence encoding the above ARSB fusion is provided.

(SEQ ID NO: 19) [symbol]
ATGGACCCCCCACG
ACCTGCTCTTCTTGCTTTGCTCGCTTTGCCCGCATTGCTCCTGCTCCTTC
TGGCGGGCGCCCGGGCCAGTGGGGCAGGAGCTTCTCGCCCACCTCACCTT
GTCTTCCTGCTTGCTGACGACCTTGGATGGAATGATGTGGGTTTTCACGG
TTCCCGGATTCGAACACCACACTTGGATGCTCTGGCTGCGGGGGGCGTAC
TGCTCGATAATTATTACACACAACCGTTGTGCACACCGTCTCGGAGTCAG
CTTCTGACGGGACGATATCAAATAAGGACTGGGCTGCAACATCAGATTAT
ATGGCCATGTCAACCCAGTTGCGTGCCCCTTGACGAGAAACTCCTGCCTC
AGCTGTTGAAGGAGGCTGGATACACGACACATATGGTCGGCAAGTGGCAT
CTGGGAATGTACAGGAAAGAGTGTCTCCCAACCCGGCGCGGATTCGATAC
GTATTTTGGGTACTTGTTGGGCTCAGAAGATTACTATTCACACGAAAGGT
GCACTCTTATCGACGCACTCAACGTTACGCGCTGCGCACTTGATTTCCGA
GATGGAGAGGAAGTTGCAACTGGGTATAAGAATATGTATTCAACAAACAT
CTTTACTAAGCGGGCAATAGCCCTCATTACTAATCATCCCCCCGAAAAAC
CGCTGTTTCTCTATCTCGCCCTGCAGTCAGTCCATGAGCCGCTGCAGGTG
CCTGAGGAGTACCTCAAACCATACGACTTCATACAAGATAAGAACCGACA
CCATTATGCGGGTATGGTCTCTCTGATGGACGAGGCTGTCGGGAATGTTA
CTGCAGCATTGAAATCTTCCGGCCTTTGGAACAACACAGTCTTCATATTT
TCTACGGACAATGGCGGGCAGACTCTTGCCGGTGGCAATAACTGGCCTCT
TAGGGGTCGCAAGTGGTCCCTCTGGGAAGGAGGCGTCCGCGGTGTAGGGT
TTGTAGCGAGCCCCCTCCTTAAACAAAAAGGAGTTAAAAACAGGGAACTT
ATTCACATTTCCGATTGGTTGCCCACGCTCGTCAAGCTCGCGAGGGGTCA
TACAAACGGGACAAAACCGCTTGACGGGTTTGATGTATGGAAAACCATTT
CTGAGGGGTCTCCTAGCCCCAGGATCGAGCTGTTGCATAACATAGACCCA
AACTTTGTAGACTCCTCACCCTGTCCCCGAAATTCAATGGCCCCTGCCAA
AGATGATTCATCCCTCCCTGAGTACTCCGCTTTTAACACAAGCGTACATG
CCGCTATTAGGCACGGCAATTGGAAGCTGCTGACCGGGTATCCTGGTTGT
GGTTATTGGTTCCCTCCCCCCTCACAATACAACGTGTCTGAAATACCGAG
TAGTGATCCGCCGACAAAAACCCTGTGGCTGTTCGATATTGATCGAGACC
CTGAAGAACGCCACGATCTCTCCCGGGAATACCCCCACATTGTCACCAAG
CTTCTTTCACGATTGCAATTTTACCACAAGCATTCAGTACCCGTTTACTT
CCCCGCCCAGGACCCAAGGTGCGATCCTAAAGCAACGGGGGTTTGGGGAC
CATGGATGCACCACCACCACCATCATTA

[symbol]

The double underlined sequence at the 5′-end and the bold italic sequence at the 3′-end represent end tag sequences that can be used for manufacturing/amplification of the coding sequences in the middle.

In certain embodiments, the GOI encodes an immunoglobulin or an antigen-binding fragment thereof that neutralizes an antigen in a host, and wherein neutralization of the antigen alleviates a symptom or a cause of a disease or condition.

In certain embodiments, the antigen is TNFα, wherein the GOI encodes adalimumab, infliximab, golimumab, ustekinumab, exemptia, adfrar, amjevita, cyltezo, idacio, inflectra, remsima, infimab, inflectra (infliximab-dyyb), renflexis (infliximab-abda), flixabi, certolizumab pegol/CDP870, etanercept, and/or wherein the disease or disorder is rheumatoid arthritis, psoriatic arthritis, ankylosing spondylitis, Crohn's disease, ulcerative colitis, psoriasis, hidradenitis suppurativa, uveitis, and juvenile idiopathic arthritis.

Another aspect of the invention provides a pharmaceutical composition comprising the rAAV vector of the invention.

Another aspect of the invention provides a cell infected with the rAAV vector of the invention.

Another aspect of the invention provides a recombinant AAV virus comprising the rAAV vector of the invention, wherein the serotype or pseudotype of the recombinant AAV virus is for preferential infection of liver or hepatic-tissue, or for preferential infection of skeletal muscle tissue.

Another aspect of the invention provides a method of treating a (genetic) disease or disorder in a subject, the method comprising introducing the recombinant AAV virus of the invention into the subject.

In certain embodiments, the recombinant AAV virus preferentially infects the liver or skeletal muscle tissue of the subject, and causes secretion of the protein encoded by the gene of interest into circulation.

Another aspect of the invention provides a method of producing the rAAV vector of the invention, comprising introducing the rAAV vector of the invention into a packaging cell line that constitutively or inducibly provides rep/cap proteins in trans.

In certain embodiments, the packaging cell line is a HEK293 cell line, a HeLa cell, or an A549 cell.

In general, the subject rAAV vector can be produced using any of many art-recognized approach. In certain embodiments, the rAAV vector is produced based on the helper-virus-free transient transfection method with all cis and trans components (e.g., vector plasmid and packaging plasmids, along with helper genes isolated from adenovirus) in host cells such as 293 cells. In certain embodiments, the rAAV vector is produced using recombinant herpes simplex virus (rHSV)-based AAV production system, which utilizes rHSV vectors to bring the AAV vector and the Rep and Cap genes into the cells. In certain embodiments, the rAAV vector is produced based on baculovirus system which requires simultaneous infection of insect cells with several baculovirus vectors to deliver the rAAV vector cassette and the Rep and Cap genes. In certain embodiments, the rAAV vector is produced based on the AAV producer cell lines derived from HeLa or A549, which stably harbored AAV Rep/cap genes. The AAV vector cassette can either be stably integrated in the host genome or be introduced by an adenovirus that contained the cassette.

### EXAMPLES

### Example 1 Secretion of Enzymes of Interest by Muscle Cells after Cell Transfection with Plasmids Comprising rAAV Virus

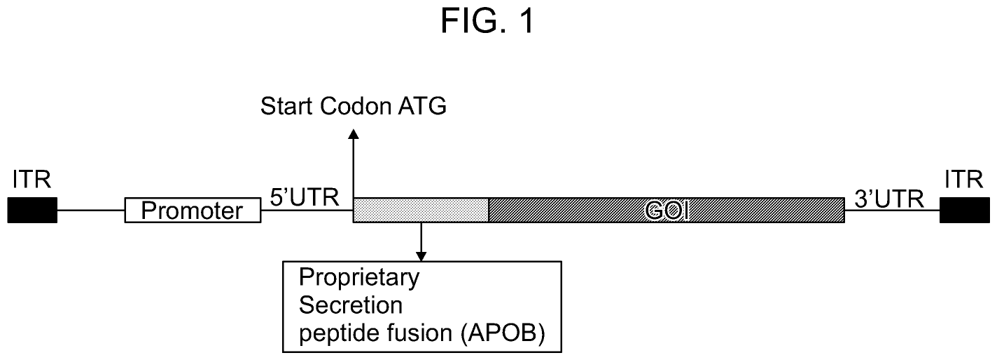

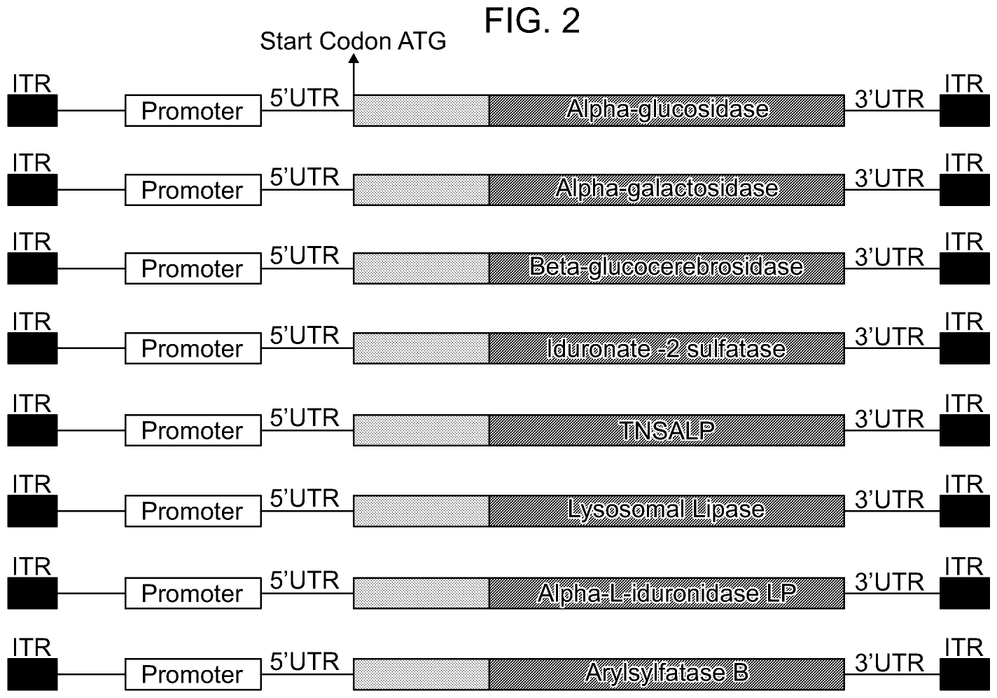

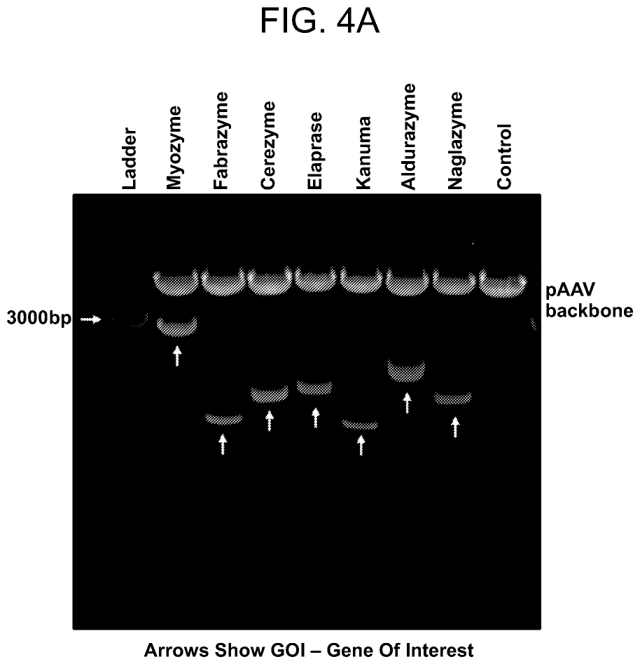

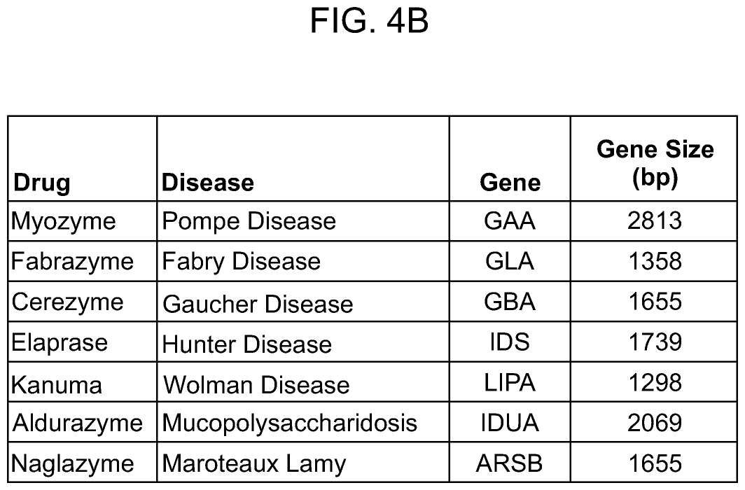

pAAV plasmids with rAAV vectors of enzymes of interest according to FIGS. 1-3 were generated. Double-cleavage of these plasmids at GOI insertion sites yielded two fragments, the pAAV backbone and the GOI fragments (FIG. 4A) at expected size. Drug names, gene names, relevant disease and sizes of the enzymes of interest are shown in FIG. 4B.

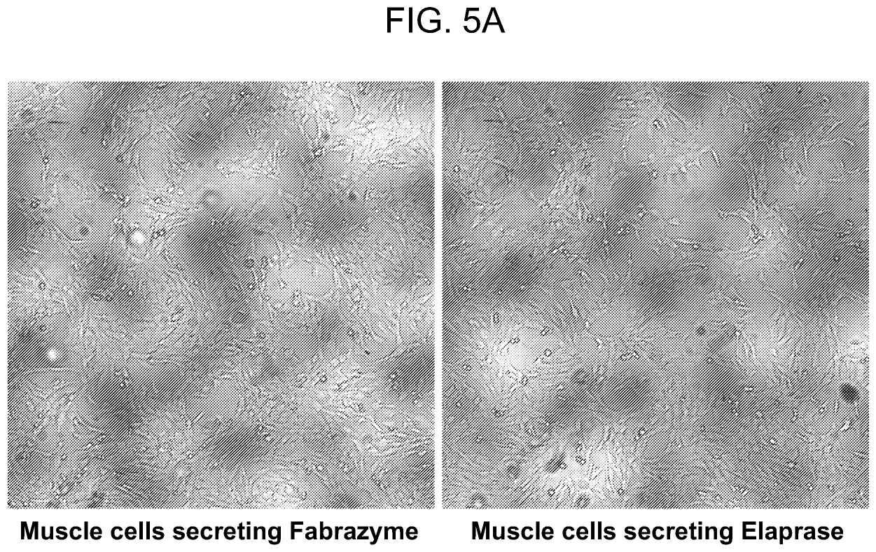

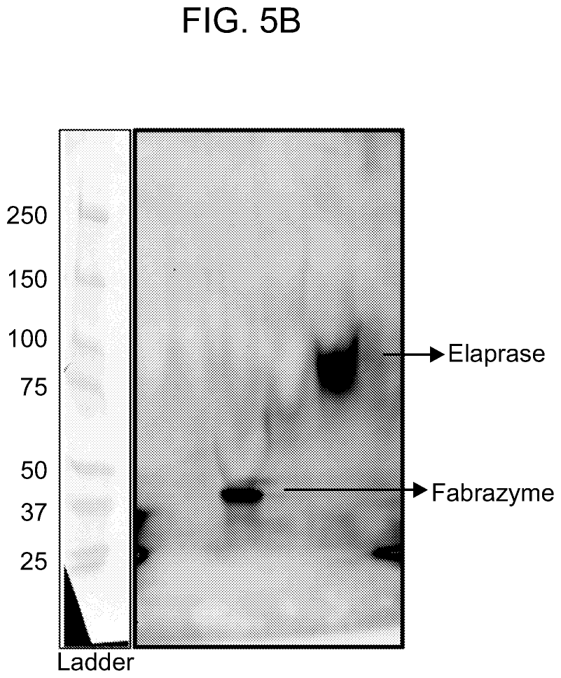

Two plasmids carrying AAV vectors for expression of Fabrazyme (GLA) and Elaprase (IDS) were tested in muscle cells. The transfected muscle cells were morphologically healthy (FIG. 5A). Using antibody against the terminal His-tag, proteins at molecular weights correlated with the sizes of Fabrazyme and Elapase were detected in the conditioned media of corresponding transduced muscle cells (FIG. 5B). This suggests that secretion of the enzymes of interest by the pAAV plasmids was both effective and not toxic to muscle cells.

### Example 2 Production of AAV Vectors Encoding Enzymes of Interest

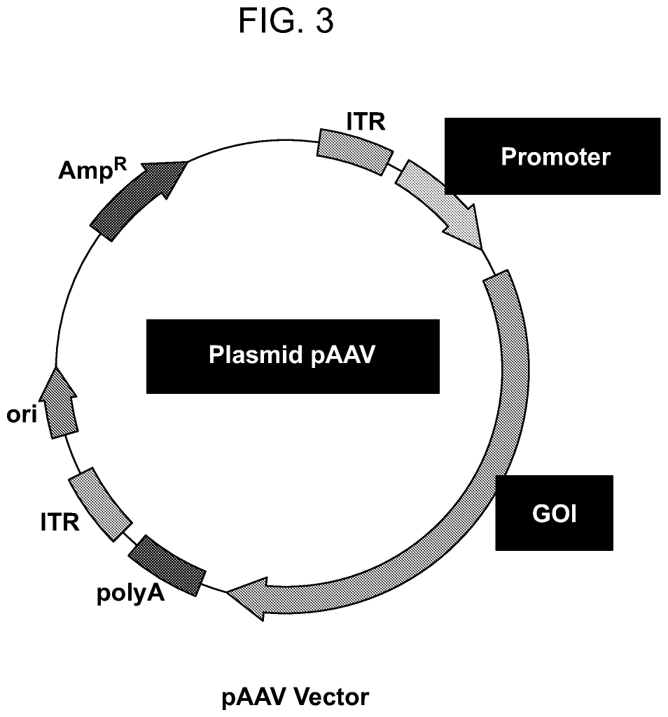

The coding sequence for one of the enzymes of interest (e.g., Fabrazyme or Elapase) is cloned into an AAV vector comprising the coding sequence under the control of a subject promoter, flanked by the AAV ITR sequences required for packaging. Such an AAV vector may be on a pAAV plasmid having the general structure of FIG. 3. The pAAV plasmid is then transfected into a suitable packaging cell line that supplies the AAV Rep and Cap proteins required for packaging AAV viral particles.

Transfection of the pAAV plasmid DNA into packaging cells such as HEK293T cells is carried out by calcium phosphate transfection, optionally with equimolar amount of the pAAV plasmids and the helper plasmid that provide the AAV Rep and Cap functions (as well as the Ad5 genes (VA RNAs, E2A, and E4OEF6)). HEK293 cells may constitutively express E1a/b, the fourth Ad function required for AAV replication. Transfected cells are amplified in Corning cellstacks or roller bottles.

Alternatively, AAV particles are produced using a two-helper method (or triple transfection), with AAV and Ad5 functions provided from separate plasmids.

Typically, up to 80% of the cells are transfected, with virus production peaking between 48-72 hours in the cell harvest.

In a typical GMP manufacturing campaign, more than a hundred cellstacks can be used for yields of about 1E15 vg of clinical product.

Alternatively, HEK293 cells adapted to grow in suspension can be used to increase production yield. Yields generated upon triple transfection with PEImax, are typically greater than 10⁵ vector genome containing particles vg/cell in crude lysates, or greater than 1×10¹⁴ vg/L of cell culture (1×10⁶ cells/mL) after 48 hours incubation.

Further alternatively, instead of using HEK293T cells, stable packaging cell lines, such as those derived from HeLa cells, are engineered by produce either the AAV rep and cap genes (packaging cell lines) and/or the rAAV genome to be produced (producer cells), in order to produce rAAV viral particles encoding the enzymes of interest.

Production of AAV viral particles can also be a baculovirus (BV) production system or HSV Type I system, such as using recombinant HSV vectors built on the replication-deficient d27.1 HSV variant that lacks ICP27 expression, and the corresponding V27 cells, a VERO-derived cell line that supports rHSV replication by providing ICP27.

Regardless of the method used for rAAV production, the obtained rAAV viral particles encoding the enzyme of interest can be dosed to mice to show secretion of the encoded enzymes in vivo, and efficacy in a mice model of the diseases due to the lack of the encoded enzyme.

For example, coding sequence for an enzyme of interest (or GOI) is fused to a proprietary muscle specific promoter of the invention, and cloned into a pAAV plasmid for producing AAV9 viral particles encompassing the coding sequence. The muscle specific AAV9 viral particles are then infused to an experimental mouse at a dose of about 5E13 or 1E14 vg.

A number of possible readouts are assessed in the mouse infused with the AAV9 encoding the enzyme of interest, including (a) secretion/detection of the encoded enzymes of interest in the serum/plasma of the mouse (e.g., via ELISA or Western blot following immunoprecipitation of the encoded enzymes); (b) detection of the enzymatic activities of the encoded enzyme in mouse serum/plasma (e.g., either isolating the encoded enzyme by the tag on the enzyme for functional assay, or isolating the enzyme using Ab specific for the enzyme in a mouse deficient for the enzyme); (c) determining the efficacy of the encoded enzymes in mice by histopathological and/or biochemical methods.

1. A recombinant adeno-associated viral (rAAV) vector, comprising:
(1) a polynucleotide encoding a secretary signal peptide fused N-terminal to a protein encoded by a gene of interest (GOI); and,
(2) a pair of inverted terminal repeat (ITR) flanking the polynucleotide;
wherein the rAAV vector has a serotype or peusotype for preferential infection of liver or skeletal muscle tissue.

2. The rAAV vector of claim 1, wherein the secretary signal peptide comprises more than 50% or 60% of hydrophobic amino acids (e.g., about 20%, 30%, 40%, or 50% Leu).

3. The rAAV vector of claim 1, wherein the secretary signal peptide comprises 5, 6, 7, 8, 9, 10, 11, 12, 13, or 14 Leu residues.

4. The rAAV vector of claim 1, wherein the secretary signal peptide is the secretary signal peptide of an albumin, a globulin, or a fibrinogen.

5. The rAAV vector of claim 1, wherein the secretary signal peptide is the secretary signal peptide of ApoB (e.g., SEQ ID NO: 1).

6. The rAAV vector of any one of claims 1-5, which expresses in vivo more than 1 μg of plasma protein/dL of blood.

7. The rAAV vector of any one of claims 1-6, wherein the serotype is AAV1, AAV6, AAV7, AAV8, or AAV9.

8. The rAAV vector of claim 7, wherein the serotype is AAV7, AAV8, or AAV9.

9. The rAAV vector of any one of claims 1-6, wherein the pseudotype is AAV2/1, AAV/2/6, AAV2/7, AAV2/8, or AAV2/9.

10. The rAAV vector of any one of claims 1-9, wherein the polynucleotide further comprises a 5′-UTR coding region, a 3′-UTR coding region, and a promoter sequence.

11. The rAAV vector of claim 10, wherein the promoter sequence is a liver- or hepatic cell-specific promoter and the serotype or peusotype is for preferential infection of liver.

12. The rAAV vector of claim 10, wherein the promoter sequence is a skeletal muscle-specific promoter and the serotype or peusotype is for preferential infection of skeletal muscle.

13. The rAAV vector of claim 12, wherein the skeletal muscle-specific promoter sequence comprises the AAVGTX sequence (SEQ ID NO: 20).

14. The rAAV vector of any one of claims 1-13, wherein the GOI encodes protein deficient in a genetic disease or disorder in a host, and wherein expression of the GOI in the host alleviates the disease or disorder.

15. The rAAV vector of claim 14, wherein the genetic disease or disorder is Pompe disease or GAA deficiency, and the GOI encodes α-glucosidase or alglucosidase alfa.

16. The rAAV vector of claim 14, wherein the genetic disease or disorder is Fabry disease or a deficiency of α-galactosidase A (α-Gal A), and the GOI encodes α-galactosidase A.

17. The rAAV vector of claim 14, wherein the genetic disease or disorder is Gaucher disease or beta-glucocerebrosidase deficiency, and the GOI encodes beta-glucocerebrosidase.

18. The rAAV vector of claim 14, wherein the genetic disease or disorder is Hunter syndrome or MPS-II, and the GOI encodes lysosomal enzyme iduronate-2-sulfatase.

19. The rAAV vector of claim 14, wherein the genetic disease or disorder is hypophosphatasia (HPP), such as perinatal/infantile- and juvenile-onset HPP, and the GOI encodes asfotase alfa.

20. The rAAV vector of claim 14, wherein the genetic disease or disorder is lysosomal acid lipase deficiency (LAL-D), and the GOI encodes lysosomal acid lipase (LAL) or sebelipase alfa.

21. The rAAV vector of claim 14, wherein the genetic disease or disorder is mucopolysaccharidosis (MPS), such as MPS type I, and the GOI encodes alpha-L-iduronidase (IDUA).

22. The rAAV vector of claim 14, wherein the genetic disease or disorder is maroteux lamy syndrome (MPS VI), and the GOI encodes arylsylfatase B (ARSB).

23. The rAAV vector of any one of claims 1-13, wherein the GOI encodes an immunoglobulin or an antigen-binding fragment thereof that neutralizes an antigen in a host, and wherein neutralization of the antigen alleviates a symptom or a cause of a disease or condition.

24. The rAAV vector of claim 23, wherein the antigen is TNFα, wherein the GOI encodes adalimumab, infliximab, golimumab, ustekinumab, exemptia, adfrar, amjevita, cyltezo, idacio, inflectra, remsima, infimab, inflectra (infliximab-dyyb), renflexis (infliximab-abda), flixabi, certolizumab pegol/CDP870, etanercept, and/or wherein the disease or disorder is rheumatoid arthritis, psoriatic arthritis, ankylosing spondylitis, Crohn's disease, ulcerative colitis, psoriasis, hidradenitis suppurativa, uveitis, and juvenile idiopathic arthritis.

25. A pharmaceutical composition comprising the rAAV vector of any one of claims 1-24.

26. A cell infected with the rAAV vector of any one of claims 1-24.

27. A recombinant AAV virus comprising the rAAV vector of any one of claims 1-24, wherein the serotype or pseudotype of the recombinant AAV virus is for preferential infection of liver or hepatic-tissue, or for preferential infection of skeletal muscle tissue.

28. A method of treating a (genetic) disease or disorder in a subject, the method comprising introducing the recombinant AAV virus of claim 24 into the subject.

29. The method of claim 28, wherein the recombinant AAV virus preferentially infects the liver or skeletal muscle tissue of the subject, and causes secretion of the protein encoded by the gene of interest into circulation.

30. A method of producing the rAAV vector of any one of claims 1-24, comprising introducing the rAAV vector of any one of claims 1-24 into a packaging cell line that constitutively or inducibly provides rep/cap proteins in trans.
